SEQUENCE LISTING (145 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 30 aa, Homo sapiens
  MOD_RES 30: note = AMIDATION
HAEGTFTSDVSSYLEGQAAKEFIAWLVKGR

SEQ ID NO: 3 - protein, 31 aa, synthetic construct
  REGION 1..31: note = Synthetic construct; Formula I
  VARIANT 1: note = Xaa at position 1 is selected from the group consisting of Y, H, D-Tyr, F, desH, and desY, or Xaa at position 1 and Xaa at position 2 combine to form desH-psi[NHCO]-Aib
  MOD_RES 1: note = The N-terminus of Xaa at position 1 is modified with R1, wherein the modification is selected from the group consisting of Ac and absent.
  VARIANT 2: note = Xaa at position 2 is selected from the group consisting of Aib, alpha-MeP, A, P, and D-Ala, or Xaa at position 1 combines with Xaa at position 2 to form desH-psi[NHCO]-Aib
  VARIANT 3: note = Xaa at position 3 is selected from the group of E, N, Aad, and cTA
  VARIANT 6: note = Xaa at position 6 is selected from the group consisting of F, alpha-MeF, and alpha-MeF(2F)
  VARIANT 10: note = Xaa at position 10 is selected from the group consisting of A, L, H, 3Pal, 4Pal, V, Y, E, alpha-MeF, alpha-MeF(2F), I, alpha-MeY, Q, D-His, D-Tyr, cTA, and K
  VARIANT 10: note = when Xaa at position 10 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  REGION 10..31: note = MISC_FEATURE - When Xaa is K(2-[2-(2-amino-ethoxy)- ethoxy]-acetyl)2-(gamma-Glu)-CO-(CH2)q-CO 2H at positions 10, 12, 13, 14, 16, 17, 19, 20, 21, 23, 24, 26, 27, 28, 29, 30 or 31, q is 14, 15, 16, 17, 18, 19 or 20.
  VARIANT 11: note = Xaa at position 11 is selected from the group consisting of S, alpha-MeS, or D-Ser
  VARIANT 12: note = Xaa at position 12 is selected from the group consisting of I, S, D-Ile, and K
  MOD_RES 12: note = when Xaa at position 12 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 13: note = Xaa at position 13 is selected from the group consisting of Nle, Aib, L, alpha-MeL, and K
  MOD_RES 13: note = when Xaa at position 13 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 14: note = Xaa at position 14 is selected from the group consisting of L and K, wherein K is conjugated to a C16-C22 fatty acid wherein said fatty acid is optionally conjugated to said K via a linker
  VARIANT 16: note = Xaa at position 16 is selected from the group consisting of E, Orn, Dab, Dap, S, T, H, Aib, alpha-MeK, R, and K
  MOD_RES 16: note = when Xaa at position 16 is K, then K is optionally chemically modified by conjugation of the epsilon-amino group of the K side chain with (2-[2-(2-amino-ethoxy)-ethoxy]-acetyl)2 -(gamma-Glu)-CO-(CH2)q-CO2H
  VARIANT 17: note = Xaa at position 17 is selected from the group consisting of K, Q, I, and an amino acid conjugated to a C16-C22 fatty acid wherein said fatty acid is optionally conjugated to said amino acid via a linker
  VARIANT 19: note = Xaa at position 19 is selected from the group consisting of Q, A, and K
  MOD_RES 19: note = when Xaa at position 19 is K, then K is chemically modified by conjugation of the epsilon-amino group of the K side chain with (2-[2-(2-amino-ethoxy)-ethoxy]-acetyl)2 -(gamma-Glu)-CO-(CH2)q-CO2H
  VARIANT 20: note = Xaa at position 20 is selected from the group consisting of Aib, Q, H, R, K, and alpha-MeK
  MOD_RES 20: note = when Xaa at position 20 is K, then K is optionally chemically modified by conjugation of the epsilon-amino group of the K side chain with (2-[2-(2-amino-ethoxy)-ethoxy]-acetyl)2 -(gamma-Glu)-CO-(CH2)q-CO2H
  VARIANT 21: note = Xaa at position 21 is selected from the group consisting of H, Aad, D, Aib, T, A, E, I, and K
  MOD_RES 21: note = when Xaa at position 21 is K, then K is chemically modified by conjugation of the epsilon-amino group of the K side chain with K(2-[2-(2-amino-ethoxy)-ethoxy]-acetyl)2 -(gamma-Glu)-CO-(CH2)q-CO 2H
  VARIANT 22: note = Xaa at position 22 is selected from the group consisting of F and alpha-MeF
  VARIANT 23: note = Xaa at position 23 is selected from the group consisting of I, L, A, G, F, H, E, V, and K
  MOD_RES 23: note = when Xaa at position 23 is K, then K is chemically modified by conjugation of the epsilon-amino group of the K side chain with K(2-[2-(2-amino-ethoxy)-ethoxy]-acetyl)2 -(gamma-Glu)-CO-(CH2)q-CO 2H
  VARIANT 24: note = Xaa at position 24 is selected from the group consisting of S, Aad, D-Glu, E, Aib, H, V, A, Q, D, P, and K
  MOD_RES 24: note = when Xaa at position 24 is K, then K is chemically modified by conjugation of the epsilon-amino group of the K side chain with K(2-[2-(2-amino-ethoxy)-ethoxy]-acetyl)2 -(gamma-Glu)-CO-(CH2)q-CO 2H
  VARIANT 25: note = Xaa at position 25 is selected from the group consisting of Y or alpha-MeY
  VARIANT 26: note = Xaa at position 26 is selected from the group consisting of L, alpha-MeL, and K
  MOD_RES 26: note = when Xaa at position 26 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 27: note = Xaa at position 27 is selected from the group consisting of L, I, and K
  MOD_RES 27: note = when Xaa at position 27 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 28: note = Xaa at position 28 is selected from the group consisting of E, A, S, D-Glu, and K
  MOD_RES 28: note = when Xaa at position 28 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 29: note = Xaa at position 29 is selected from the group consisting of Aib, G, A, and K
  MOD_RES 29: note = when Xaa at position 29 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 30: note = Xaa at position 30 is selected from the group consisting of C, G, G-R2, and K, wherein R2 is a modification of the C-terminal group, wherein the modification is NH2 or absent, wherein if X30 is G-R2, then X31 is absent.
  MOD_RES 30: note = when Xaa at position 30 is K, then K is chemically modified by conjugation of the epsilon-amino group of the K side chain with (2-[2-(2-amino-ethoxy)-ethoxy]-acetyl)2- (gamma-Glu)-CO-(CH2)q-CO2 H
  VARIANT 31: note = Xaa at position 31 is absent or is selected from the group consisting of SEQ ID NO:4, SEQ ID NO:5, SEQ ID:6, SEQ ID NO:7, SEQ ID NO:8, and SEQ ID NO:9
  SITE 31: note = MISC_FEATURE - wherein no more than one of X10, X12, X13, X14, X16, X17, X19, X20, X21, X23, X24, X26, X27, X28, X29, X30, X31, X32, X33, X34, X35, X36, X37, X38, X39, and X40 may be a substituent that contains a fatty acid
  SITE 31: note = MISC_FEATURE - wherein no more than one of X30, X34, X39, and X40 may be C
  SITE 31: note = MISC_FEATURE - wherein if one of X30, X34, X39, and X40 is C, then none of X10, X12, X13, X14, X16, X17, X19, X20, X21, X23, X24, X26, X27, X28, X29, X30, X31, X32, X33, X34, X35, X36, X37, X38, X39, and X40 is a substituent that contains a fatty acid
  MOD_RES 31: note = Xaa at position 31 is modified with R2, wherein the modification is NH2 to form a C-terminal amide or absent
XXXGTXTSDXXXXXDXXAXXXXXXXXXXXXX

SEQ ID NO: 4-9 - skipped (6 entries)

SEQ ID NO: 10 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-MeF(2F)
  SITE 13: note = MISC_FEATURE - Xaa at position 13 is alpha-MeL
  MOD_RES 17: note = K at position 17 is chemically modified by conjugation of the epsilon-amino group of the K side chain with (2-[2-(2-amino-ethoxy)-ethoxy]-acetyl)2-(gamma-Glu)-C O-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = The Serine at position 39 is amidated
YXEGTXTSDYSIXLDEKAQXEFIXYLIEGGPSSGAPPPS

SEQ ID NO: 11 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-MeF(2F)
  SITE 13: note = MISC_FEATURE - Xaa at position 13 is alpha-MeL
  SITE 16: note = MISC_FEATURE - Xaa at position 16 is Orn
  MOD_RES 17: note = K at position 17 is chemically modified by conjugation of the epsilon-amino group of the K side chain with (2-[2-(2-amino-ethoxy)-ethoxy]-acetyl)2-(gamma-Glu)-C O-(CH2)16-CO2H
  SITE 20: note = MISC_FEATURE - Xaa at position 20 is Aib
  SITE 24: note = MISC_FEATURE - Xaa at position 24 is D-Glu
  MOD_RES 39: note = The Serine at position 39 is amidated
YXEGTXTSDYSIXLDXKAQXEFIXYLIEGGPSSGAPPPS

SEQ ID NO: 12 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-MeF(2F)
  SITE 13: note = MISC_FEATURE - Xaa at position 13 is alpha-MeL
  SITE 16: note = MISC_FEATURE - Xaa at position 16 is Orn
  MOD_RES 17: note = K at position 17 is chemically modified by conjugation of the epsilon-amino group of the K side chain with (2-[2-(2-amino-ethoxy)-ethoxy]-acetyl)2-(gamma-Glu)-C O-(CH2)18-CO 2H
  SITE 20: note = MISC_FEATURE - Xaa at position 20 is Aib
  SITE 24: note = MISC_FEATURE - Xaa at position 24 is D-Glu
  SITE 39: note = MISC_FEATURE - The Serine at position 39 is amidated

SEQ ID NO: 13 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-MeF(2F)
  SITE 10: note = MISC_FEATURE - Xaa at position 10 is 4Pal
  SITE 13: note = MISC_FEATURE - Xaa at position 13 is alpha-MeL
  SITE 16: note = MISC_FEATURE - Xaa at position 16 is Orn
  MOD_RES 17: note = K at position 17 is chemically modified by conjugation of the epsilon-amino group of the K side chain with (2-[2-(2-amino-ethoxy)-ethoxy]-acetyl)2-(gamma-Glu)-C O-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-MeY
  MOD_RES 39: note = The Serine at position 39 is amidated
YXEGTXTSDXSIXLDXKAQXEFIXXLIEGGPSSGAPPPS

SEQ ID NO: 14 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKKAQXAFIEYLIEGGPSSGAPPPS

SEQ ID NO: 15 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDSKAQXAFIEYLIEGGPSSGAPPPS

SEQ ID NO: 16 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDKKAQXAFIEYLIEGGPSSGAPPPS

SEQ ID NO: 17 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is alpha-methyl-Lys
  MOD_RES 39: note = Ser at position 39 is amidated
YXEGTFTSDYSIXLDEKAQXAFIEYLLEGGPSSGAPPPS

SEQ ID NO: 18 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is alpha-methyl-Lys
  SITE 29: note = MISC_FEATURE - Xaa at position 29 is Aib
  MOD_RES 39: note = Ser at position 39 is amidated
YXEGTFTSDYSIXLDEKAQXAFIEYLLEXGPSSGAPPPS

SEQ ID NO: 19 - protein, 39 aa, synthetic construct
  REGION 1..39: note = Synthetic Construct
  SITE 2: note = MISC_FEATURE - Xaa at position 2 is Aib
  MOD_RES 12: note = Lys at position 12 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSKLLDKIAQXAFIEYLIEGGPSSGAPPPS

SEQ ID NO: 20 - protein, 39 aa, synthetic construct
  REGION 1..39: note = Synthetic Construct
  SITE 2: note = MISC_FEATURE - Xaa at position 2 is Aib
  MOD_RES 13: note = Lys at position 13 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIKLDKIAQXAFIEYLIEGGPSSGAPPPS

SEQ ID NO: 21 - protein, 39 aa, synthetic construct
  REGION 1..39: note = Synthetic Construct
  SITE 2: note = MISC_FEATURE - Xaa at position 2 is Aib
  MOD_RES 16: note = Lys at position 16 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKIAQXAFIEYLIEGGPSSGAPPPS

SEQ ID NO: 22 - protein, 39 aa, synthetic construct
  REGION 1..39: note = Synthetic Construct
  SITE 2: note = MISC_FEATURE - Xaa at position 2 is Aib
  MOD_RES 19: note = Lys at position 19 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSILLDKIAKXAFIEYLIEGGPSSGAPPPS

SEQ ID NO: 23 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 21: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXKFIEYLIEGGPSSGAPPPS

SEQ ID NO: 24 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 23: note = Lys at position 23 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFKEYLIEGGPSSGAPPPS

SEQ ID NO: 25 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 24: note = Lys at position 24 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIKYLIEGGPSSGAPPPS

SEQ ID NO: 26 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 28: note = Lys at position 28 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIKGGPSSGAPPPS

SEQ ID NO: 27 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 32: note = Lys at position 32 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPKSGAPPPS

SEQ ID NO: 28 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 37: note = Lys at position 37 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPSSGAPKPS

SEQ ID NO: 29 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 38: note = Lys at position 38 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Ser at position 39 is amidated
YXEGTFTSDYSILLDKIAQXAFIEYLIEGGPSSGAPPKS

SEQ ID NO: 30 - protein, 39 aa, synthetic construct
  REGION 1..39: note = Synthetic Construct
  SITE 2: note = MISC_FEATURE - Xaa at position 2 is Aib
  SITE 20: note = MISC_FEATURE - Xaa at position 20 is Aib
  MOD_RES 39: note = Lys at position 39 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 39: note = Lys at position 39 is amidated
YXEGTFTSDYSILLDKIAQXAFIEYLIEGGPSSGAPPPK

SEQ ID NO: 31 - protein, 34 aa, synthetic construct
  REGION 1..34: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 34: note = Gly at position 34 is amidated
YXEGTFTSDYSILLDKKAQXAFIEYLIEGGPSSG

SEQ ID NO: 32 - protein, 30 aa, synthetic construct
  REGION 1..30: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSILLDKKAQXAFIEYLIEGG

SEQ ID NO: 33 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDSKAQXAFIEYLIEGGPSSGAPPPS

SEQ ID NO: 34 - protein, 39 aa, synthetic construct
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  SITE 16: note = MISC_FEATURE - Xaa at position 16 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYSIXLDXKAQXAFIEYLIEGGPSSGAPPPS

SEQ ID NO: 35 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSIXLDKKAQXAFIEYLIEGG

SEQ ID NO: 36 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSIXLDEKAQXAFIEYLIEGG

SEQ ID NO: 37 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSIXLDSKAQXAFIEYLIEGG

SEQ ID NO: 38 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTFTSDYSIXLDTKAQXAFIEYLIEGG

SEQ ID NO: 39 - protein, 39 aa, synthetic construct; identical to SEQ ID NO: 17
  REGION 1..39: note = Synthetic Construct
  SITE 2: note = MISC_FEATURE - Xaa at position 2 is Aib
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 40 - protein, 39 aa, synthetic construct
  REGION 1..39: note = Synthetic Construct
  SITE 2: note = MISC_FEATURE - Xaa at position 2 is Aib
  SITE 11: note = MISC_FEATURE - Xaa at position 11 is alpha-methyl-Ser
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTFTSDYXILLDKKAQXAFIEYLIEGGPSSGAPPPS

SEQ ID NO: 41 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXAFIEYLIEGGPSSGAPPPS

SEQ ID NO: 42 - protein, 30 aa, synthetic construct
  REGION 1..30: note = Synthetic Construct
  SITE 2: note = MISC_FEATURE - Xaa at position 2 is Aib
  SITE 20: note = MISC_FEATURE - Xaa at position 20 is Aib
  MOD_RES 28: note = Lys at position 28 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 30: note = Gly at position 30 is amidated
YXEGTFTSDYSILLDKIAQXAFIEYLIKGG

SEQ ID NO: 43 - protein, 39 aa, synthetic construct; identical to SEQ ID NO: 14
  REGION 1..39: note = Synthetic Construct
  SITE 2: note = MISC_FEATURE - Xaa at position 2 is D-Ala
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 44 - protein, 39 aa, synthetic construct; identical to SEQ ID NO: 40
  REGION 1..39: note = Synthetic Construct
  SITE 2: note = MISC_FEATURE - Xaa at position 2 is Aib
  SITE 11: note = MISC_FEATURE - Xaa at position 11 is D-Ser
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 45 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 28: note = MISC_FEATURE - Xaa at position 28 is D-Glu
  MOD_RES 39: note = Ser at position 39 is amidated
YXEGTFTSDYSILLDKKAQXAFIEYLIXGGPSSGAPPPS

SEQ ID NO: 46 - protein, 39 aa, synthetic construct
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTFTSDYSILLDKKAQXAFIXYLIEGGPSSGAPPPS

SEQ ID NO: 47 - protein, 39 aa, synthetic construct
  REGION 1..39: note = Synthetic Construct
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YPEGTFTSDYSILLDKKAQXAFIEYLIEGGPSSGAPPPS

SEQ ID NO: 48 - protein, 39 aa, synthetic construct
  REGION 1..39: note = Synthetic Construct
  SITE 2: note = MISC_FEATURE - Xaa at position 2 is Aib
  SITE 3: note = MISC_FEATURE - Xaa at position 3 is Aad
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXXGTFTSDYSILLDKKAQXAFIEYLIEGGPSSGAPPPS

SEQ ID NO: 49 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 20: note = MISC_FEATURE - Xaa at position 20 is Aib
  MOD_RES 28: note = Lys at position 28 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 30: note = Gly at position 30 is amidated
YXEGTXTSDYSILLDKIAQXAFIEYLIKGG

SEQ ID NO: 50 - protein, 30 aa, synthetic construct
  REGION 1..30: note = Synthetic Construct
  SITE 2: note = MISC_FEATURE - Xaa at position 2 is Aib
  SITE 13: note = MISC_FEATURE - Xaa at position 13 is alpha-methyl-Leu
  SITE 20: note = MISC_FEATURE - Xaa at position 20 is Aib
  MOD_RES 28: note = Lys at position 28 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  MOD_RES 30: note = Gly at position 30 is amidated
YXEGTFTSDYSIXLDKIAQXAFIEYLIKGG

SEQ ID NO: 51 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 13 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTXTSDYSILLDKKAQXAFIEYLIEGG

SEQ ID NO: 52 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTXTSDYSIXLDKKAQXAFIEYLIEGG

SEQ ID NO: 53 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 30: note = Gly at position 30 is amidated
YXEGTXTSDYSIXLDEKAQXEFIEYLIEGG

SEQ ID NO: 54 - protein, 30 aa, synthetic construct; identical to SEQ ID NO: 53
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  MOD_RES 30: note = Gly at position 30 is amidated

SEQ ID NO: 55 - protein, 39 aa, synthetic construct; identical to SEQ ID NO: 14
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-AOC-(gam ma-Glu)-CO-(CH2)18 -CO2H
  SITE 20: note = MISC_FEATURE - Xaa at position 20 is Aib
  MOD_RES 39: note = Ser at position 39 is amidated

SEQ ID NO: 56 - protein, 39 aa, synthetic construct; identical to SEQ ID NO: 14
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with AOC-(2-[2-(2-amino-ethoxy)-ethoxy]-acetyl)-(gam ma-Glu)-CO-(CH2)18 -CO2H
  SITE 20: note = MISC_FEATURE - Xaa at position 20 is Aib
  MOD_RES 39: note = Ser at position 39 is amidated

SEQ ID NO: 57 - protein, 39 aa, synthetic construct; identical to SEQ ID NO: 14
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-(gamma-G lu)-(Trx)-CO-(CH2) 18-CO2H
  SITE 20: note = MISC_FEATURE - Xaa at position 20 is Aib
  MOD_RES 39: note = Ser at position 39 is amidated

SEQ ID NO: 58 - protein, 39 aa, synthetic construct; identical to SEQ ID NO: 14
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-(Trx)-(g amma-Glu)-CO-(CH2) 18-CO2H
  SITE 20: note = MISC_FEATURE - Xaa at position 20 is Aib
  MOD_RES 39: note = Ser at position 39 is amidated

SEQ ID NO: 59 - protein, 39 aa, synthetic construct; identical to SEQ ID NO: 14
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-(epsilon -Lys)-(gamma-Glu)- CO-(CH2)18-CO2H
  SITE 20: note = MISC_FEATURE - Xaa at position 20 is Aib
  MOD_RES 39: note = Ser at position 39 is amidated

SEQ ID NO: 60 - protein, 39 aa, synthetic construct; identical to SEQ ID NO: 14
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)18- CO2H
  SITE 20: note = MISC_FEATURE - Xaa at position 20 is Aib
  MOD_RES 39: note = Ser at position 39 is amidated

SEQ ID NO: 61 - protein, 39 aa, synthetic construct; identical to SEQ ID NO: 14
  REGION 1..39: note = Synthetic Construct
  SITE 2: note = MISC_FEATURE - Xaa at position 2 is Aib
  MOD_RES 17: note = Lys at position 17 is chemically modified by conjugation of the epsilon-amino group of the Lys side chain with (2-[2-(2-amino-ethoxy)-ethoxy]-acetyl)-(epsilon -Lys)-(epsilon-Lys )-CO-(CH2)18-CO2H
  SITE 20: note = MISC_FEATURE - Xaa at position 2 is Aib
  MOD_RES 39: note = Ser at position 39 is amidated

SEQ ID NO: 62 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXAFIEYLIEGGPSSGAPPPS

SEQ ID NO: 63 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIEYLIEGGPSSGAPPPS

SEQ ID NO: 64 - protein, 39 aa, synthetic construct
  REGION 1..39: note = Synthetic Construct
  SITE 2: note = MISC_FEATURE - Xaa at position 2 is Aib
  SITE 3: note = MISC_FEATURE - Xaa at position 3 is cTA
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXXGTXTSDYSIXLDEKAQXAFIEYLIEGGPSSGAPPPS

SEQ ID NO: 65 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 34: note = Gly at position 34 is amidated
YXEGTXTSDYSIXLDKKAQXEFIEYLIEGGPSSG

SEQ ID NO: 66 - protein, 39 aa, synthetic construct; identical to SEQ ID NO: 41
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)2-CO-(CH2)18-C O2H
  SITE 20: note = MISC_FEATURE - Xaa at position 20 is Aib
  MOD_RES 39: note = Ser at position 39 is amidated

SEQ ID NO: 67 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXAFIEYLIEAGPSSGAPPPS

SEQ ID NO: 68 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDHSILLDKKAQXAFIEYLIEGGPSSGAPPPS

SEQ ID NO: 69 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDLSILLDKKAQXAFIEYLIEGGPSSGAPPPS

SEQ ID NO: 70 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 21: note = MISC_FEATURE - Xaa at position 21 is Aad
  MOD_RES 39: note = Ser at position 39 is amidated
YXEGTXTSDYSIXLDKKAQXXFIEYLIEGGPSSGAPPPS

SEQ ID NO: 71 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXTFIEYLIEGGPSSGAPPPS

SEQ ID NO: 72 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDKKAQXDFIEYLIEGGPSSGAPPPS

SEQ ID NO: 73 - protein, 39 aa, synthetic construct; identical to SEQ ID NO: 70
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 21: note = MISC_FEATURE - Xaa at position 21 is Aib
  MOD_RES 39: note = Ser at position 39 is amidated

SEQ ID NO: 74 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 21: note = MISC_FEATURE - Xaa at position 21 is Aib
  MOD_RES 39: note = Ser at position 39 is amidated
YXEGTXTSDYSIXLDKKAQHXFIEYLIEGGPSSGAPPPS

SEQ ID NO: 75 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is Aad
  MOD_RES 39: note = Ser at position 39 is amidated

SEQ ID NO: 76 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIAYLIEGGPSSGAPPPS

SEQ ID NO: 77 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIVYLIEGGPSSGAPPPS

SEQ ID NO: 78 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFISYLIEGGPSSGAPPPS

SEQ ID NO: 79 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIPYLIEGGPSSGAPPPS

SEQ ID NO: 80 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is Aib
  MOD_RES 39: note = Ser at position 39 is amidated

SEQ ID NO: 81 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDYSIXLDEKAQXEFIHYLIEGGPSSGAPPPS

SEQ ID NO: 82 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDHSIXLDEKAQXEFIEYLIEGGPSSGAPPPS

SEQ ID NO: 83 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is cTA
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated
YXEGTXTSDXSIXLDEKAQXEFIEYLIEGGPSSGAPPPS

SEQ ID NO: 84 - protein, 39 aa, synthetic construct; identical to SEQ ID NO: 83
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 3Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  MOD_RES 39: note = Ser at position 39 is amidated

SEQ ID NO: 85 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated
YXEGTXTSDHSIXLDEKAQXTFIXYLIEGGPSSG

SEQ ID NO: 86 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDHSIXLDEKAQXTFIXYLIEGGPSSGAPPPS

SEQ ID NO: 87 - protein, 39 aa, synthetic construct; identical to SEQ ID NO: 86
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 88 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDHSIXLDXKAQXTFIXYLIEGGPSSGAPPPS

SEQ ID NO: 89 - protein, 39 aa, synthetic construct; identical to SEQ ID NO: 88
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is a Aib
  SITE 24: note = MISC_FEATURE - Xaa at position 24 is D-Glu
  MOD_RES 39: note = Ser at position 39 is amidated

SEQ ID NO: 90 - protein, 39 aa, synthetic construct; identical to SEQ ID NO: 88
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dap
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 91 - protein, 39 aa, synthetic construct; identical to SEQ ID NO: 86
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 92 - protein, 39 aa, synthetic construct; identical to SEQ ID NO: 86
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)16- CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 93 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)14-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDHSIXLDEKAQXHFIXYLIEGGPSSGAPPPS

SEQ ID NO: 94 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated
YXEGTXTSDHSIXLDEKAQXHFIXYLIEGGPSSG

SEQ ID NO: 95 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 30: note = Gly at position 30 is amidated
YXEGTXTSDHSIXLDEKAQXHFIXYLIEGG

SEQ ID NO: 96 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 3Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDXSIXLDEKAQXEFIXYLIEGGPSSGAPPPS

SEQ ID NO: 97 - protein, 39 aa, synthetic construct; identical to SEQ ID NO: 96
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 98 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDLSIXLDEKAQXEFIXYLIEGGPSSGAPPPS

SEQ ID NO: 99 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDQSIXLDEKAQXEFIXYLIEGGPSSGAPPPS

SEQ ID NO: 100 - protein, 39 aa, synthetic construct; identical to SEQ ID NO: 96
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is Aib
  MOD_RES 39: note = Ser at position 39 is amidated

SEQ ID NO: 101 - protein, 34 aa, synthetic construct
  REGION 1..34: note = Synthetic Construct
  SITE 2: note = MISC_FEATURE - Xaa at position 2 is alpha-methyl-Pro
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  MOD_RES 34: note = Gly at position 34 is amidated
YXEGTFTSDYSILLDEKAQQAFIEYLIEGGPSSG

SEQ ID NO: 102 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-Amino-ethoxy)-ethoxy]-acetyl)-(gamma-G lu)-(2-[2-(2-Amino -ethoxy)-ethoxy]-acetyl)-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDXSIXLDKKAQXEFIXYLIEGGPSSGAPPPS

SEQ ID NO: 103 - protein, 39 aa, synthetic construct; identical to SEQ ID NO: 102
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 104 - protein, 39 aa, synthetic construct; identical to SEQ ID NO: 96
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 105 - protein, 39 aa, synthetic construct; identical to SEQ ID NO: 96
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)16-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 106 - protein, 39 aa, synthetic construct; identical to SEQ ID NO: 96
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 107 - protein, 39 aa, synthetic construct; identical to SEQ ID NO: 96
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-Amino-ethoxy)-ethoxy]-acetyl)2-(epsilo n-Lys)-CO-(CH2)18- CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 108 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)14-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 109 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 110 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dap
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 111 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-Amino-ethoxy)-ethoxy]-acetyl)-(gamma-G lu)-(2-[2-(2-Amino -ethoxy)-ethoxy]-acetyl)-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSIXLDKKAQXEFIXYLIEGGPSSGAPPPS

SEQ ID NO: 112 - protein, 39 aa, synthetic construct; identical to SEQ ID NO: 111
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 113 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 114 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)16-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 115 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-CO-(CH2)18-CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 116 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)18- CO2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 117 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 118 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Dap
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 119 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated
YXEGTXTSDYSIXLDEKAQXEFIXYLIEGGPSSG

SEQ ID NO: 120 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 30: note = Gly at position 30 is amidated
YXEGTXTSDYSIXLDEKAQXEFIXYLIEGG

SEQ ID NO: 121 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 122 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 alpha-methyl-Tyr
  MOD_RES 39: note = Ser at position 39 is amidated
YXEGTXTSDYSIXLDEKAQXEFIXXLIEGGPSSGAPPPS

SEQ ID NO: 123 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 25: note = MISC_FEATURE - Xaa at position 25 is alpha-methyl-Tyr
  MOD_RES 39: note = Ser at position 39 is amidated
YXEGTXTSDYSIXLDEKAQXEFIEXLIEGGPSSGAPPPS

SEQ ID NO: 124 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 125 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is Aib
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 126 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSILLDEKAQXEFIXYLIEGGPSSGAPPPS

SEQ ID NO: 127 - protein, 39 aa, synthetic construct; identical to SEQ ID NO: 10
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 128 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 34: note = Gly at position 34 is amidated
YXEGTXTSDYSIXLDKKAQXEFIXYLIEGGPSSG

SEQ ID NO: 129 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSIXLDHKAQXEFIXYLIEGGPSSGAPPPS

SEQ ID NO: 130 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Dab
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDXSIXLDXKAQXEFIXYLIEGGPSSGAPPPS

SEQ ID NO: 131 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDYSIXLDXKAQXEFIXXLIEGGPSSGAPPPS

SEQ ID NO: 132 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDLSIXLDXKAQXEFIXXLIEGGPSSGAPPPS

SEQ ID NO: 133 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDXSIXLDXKAQXTFIXXLIEGGPSSGAPPPS

SEQ ID NO: 134 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDVSIXLDXKAQXTFIXXLIEGGPSSGAPPPS

SEQ ID NO: 135 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDVSIXLDXKAQXAFIXXLIEGGPSSGAPPPS

SEQ ID NO: 136 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDLSIXLDXKAQXTFIXXLIEGGPSSGAPPPS

SEQ ID NO: 137 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDLSIXLDXKAQXAFIXXLIEGGPSSGAPPPS

SEQ ID NO: 138 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDASIXLDXKAQXAFIXXLIEGGPSSGAPPPS

SEQ ID NO: 139 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is Aib
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 140 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated
YXEGTXTSDYSILLDXKAQXEFIXYLIEGGPSSGAPPPS

SEQ ID NO: 141 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is Nle
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 142 - protein, 39 aa, synthetic construct; identical to SEQ ID NO: 11
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 13: note = MISC_FEATURE - Xaa at position 13 is Aib
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)18-CO 2H
  SITE 20: note = MISC_FEATURE - Xaa at position 20 is Aib
  SITE 24: note = MISC_FEATURE - Xaa at position 24 is D-Glu
  MOD_RES 39: note = Ser at position 39 is amidated

SEQ ID NO: 143 - protein, 39 aa, synthetic construct
  REGION 1..39: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(epsilo n-Lys)-CO-(CH2)18- CO2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 39: note = Ser at position 39 is amidated
YXEGTXTSDXSIXLDXKAQXAFIXXLIEGGPSSGAPPPS

SEQ ID NO: 144 - protein, 34 aa, synthetic construct
  REGION 1..34: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 16 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 34: note = Gly at position 34 is amidated
YXEGTXTSDXSIXLDXKAQXEFIXXLIEGGPSSG

SEQ ID NO: 145 - protein, 30 aa, synthetic construct
  REGION 1..30: note = Synthetic Construct
  SITE 2: note = MISC_FEATURE - Xaa at position 2 is Aib
  SITE 6: note = MISC_FEATURE - Xaa at position 6 is alpha-methyl-Phe(2F)
  SITE 10: note = MISC_FEATURE - Xaa at position 10 is 4Pal
  SITE 13: note = MISC_FEATURE - Xaa at position 13 is alpha-methyl-Leu
  SITE 16: note = MISC_FEATURE - Xaa at position 13 is Orn
  MOD_RES 17: note = Lys at position 17 is chemically modified by conjugation of the epsilon-amino group of the Lys side chain with (2-[2-(2-amino-ethoxy)-ethoxy]-acetyl)2-(gamma- Glu)-CO-(CH2)16-CO 2H
  SITE 20: note = MISC_FEATURE - Xaa at position 20 is Aib
  SITE 24: note = MISC_FEATURE - Xaa at position 24 is D-Glu
  SITE 25: note = MISC_FEATURE - Xaa at position 25 is alpha-methyl-Tyr
  MOD_RES 30: note = Gly at position 30 is amidated
YXEGTXTSDXSIXLDXKAQXEFIXXLIEGG

(19) United States
(12) Patent Application Publication
(10) Pub. No.: US 2026/0345735 A1
(43) Pub. Date: Nov. 5, 2026

(54) METHOD OF USING A GIP/GLP1 CO-AGONIST FOR DIABETES

(71) Applicant: ELI LILLY AND COMPANY, Indianapolis, IN (US)

(72) Inventors: Jorge ALSINA-FERNANDEZ, Indianapolis, IN (US); Over CABRERA, Carmel, IN (US); Tamer COSKUN, Carmel, IN (US); Charles T. BENSON, San Diego, CA (US); Axel HAUPT, Schliersee (DE); Melissa Kay THOMAS, Indianapolis, IN (US); Shweta URVA, Carmel, IN (US)

(21) Appl. No.: 19/333,087

(22) Filed: Sep. 18, 2025

Related U.S. Application Data
(63) Continuation of application No. 19/098,553, filed on Apr. 2, 2025, now Pat. No. 12,453,758.
Application No. 19/098,553 is a division of application No. 17/260,279, filed on Jan. 14, 2021, now Pat. No. 12,295,987.

(51) Int. Cl. A61K 38/26; A61P 3/10

(52) U.S. Cl. CPC A61K 38/26; A61P 3/10

(57) ABSTRACT

The present invention provides methods of treating type 2 diabetes (T2D) using a novel dosing regimen of a GIP: GLP-1 Peptide having a GIP: GLP-1 receptor agonist potency ratio that is about 2.5:1 to about 10:1 GIP to GLP-1. Furthermore, the present invention provides methods of treating T2D using a novel dosing regimen of a GIP: GLP-1 Peptide having a GIP: GLP-1 receptor agonist potency ratio that is about 2.5:1 to about 5:1 GIP to GLP-1. Also, the present invention provides methods of inducing T2D remission using a novel dosing regimen of a GIP: GLP-1 Peptide. The present invention also provides methods of treating obesity using a novel dosing regimen of a GIP: GLP-1 Peptide.

The present application is being filed along with a Sequence Listing in ST.26 XMLformat. The Sequence Listing is provided as a file titled “X21834A_US” created Jul. 8, 2025, and is 563,941 bytes in size. The Sequence Listing in formation in the ST.26 XML format is incorporated herein by reference in its entirety.

The present invention provides methods of treating type 2 diabetes (T2D) using a novel dosing regimen of a GIP/GLP-1 co-agonist compound (hereafter GIP: GLP-1 Peptide) having a GIP: GLP-1 receptor agonist potency ratio that is about 2.5 to about 10:1 GIP to GLP-1. Furthermore, the present invention provides methods of treating T2D using a novel dosing regimen of a GIP: GLP-1 Peptide having a GIP: GLP-1 receptor agonist potency ratio that is about 2.5:1 to about 5:1 GIP to GLP-1. Also, the present invention provides methods of inducing T2D remission using a novel dosing regimen of a GIP: GLP-1 Peptide. The present invention also provides methods of treating obesity using a novel dosing regimen of a GIP: GLP-1 Peptide.

Over the past several decades, the prevalence of diabetes has continued to rise. T2DM is the most common form of diabetes accounting for approximately 90% of all diabetes. T2DM is characterized by high blood glucose levels associated mainly with insulin resistance. T2D is epidemic. Long-term consequences of T2D translate into enormous human suffering and economic costs; however, much of the morbidity associated with long-term microvascular and neuropathic complications can be substantially reduced by interventions that achieve glucose levels close to the nondiabetic range. Although new classes of medications and numerous combinations have been demonstrated to lower glycemia, it is reported that current-day management generally fails to achieve and maintain the glycemic levels most likely to provide optimal healthcare status for people with diabetes. The Medical Management of Hyperglycemia inType 2 Diabetes: A Consensus Algorithm for the Initiation and Adjustment of Therapy, DIABETES CARE, VOLUME 32:193-203; NUMBER 1, January 2009. The American Diabetes Association guidelines recommend to use HbA1c in the range of 5.7 to 6.1% (39-47 mmol/mol) as the prediabetes level. (39-47 mmol/mol). American Diabetes Association, Diabetes Care. 2018 January; 41 (Supplement 1): S55-S64. There is a significant need for a treatment method to enable patients with T2D to reach their glycemic treatment goals.

It is well-known that GLP1 treatments are associated with nausea, vomiting, and/or diarrhea. For example, one study reported that all GLP-1 receptor agonist dosing regimens significantly increased the incidence of gastrointestinal adverse events. Diabetes Technol Ther. 2015 January; 17 (1): 35-42.

Although endogenous GIP exerts strong insulinotropic effects in healthy subjects, the severe reduction in insulinotropic effect of GIP and the GIP-dependent enhancement of postprandial glucagon response have discouraged development of GIP-based therapies for T2D. Seino, et. al., GIP and GLP-1, the two incretin hormones: Similarities and differences; Journal of Diabetes Investigation, Volume 1 Issue 1/2 (February/April 2010) (8-23) p 16.

Also, previous clinical trials of a GIP/GLP1 co-agonist compound having a balanced GIP/GLP1 potency have been performed and found that tolerability at high doses was limited by gastrointestinal adverse events. Portron, A. et al. “Pharmacodynamics, pharmacokinetics, safety and tolerability of the novel dual glucose-dependent insulinotropic polypeptide/glucagon-like peptide-1 agonist RG7697 after single subcutaneous administration in healthy subjects.” Diabetes Obes. Metab. 2017; 19:1446-1453. Finan, B. et al. “Unimolecular dual incretins maximize metabolic benefits in rodents, monkeys, and humans.” Sci Trans Med. 2013; 5 (209): 209ra151. The dose limitation associated with gastrointestinal adverse events may prevent dosing to the desired effective dose, may compromise patient compliance with treatment, and may limit the effectiveness of the treatment regimen.

While treatments for T2D include GLP-1 receptor agonists, there are currently no approved T2D treatments reporting GIP/GLP receptor co-agonism. Furthermore, there are no available treatments having a ratio that is about 2.5:1 to about 10:1 GIP to GLP-1 receptor agonist potency to treat T2D.

One GIP/GLP-1 receptor co-agonist having a potency ratio of about 3.6:1 GIP: GLP-1 is known as tirzepatide. In a Phase II clinical trial, tirzepatide treatment using once weekly subcutaneous doses including a 15 mg dose provided dramatic reduction of HbA1c, remission of diabetes for many patients after 26 weeks, and dramatic improvement in weight control.

Obesity is a complex medical disorder resulting in excessive accumulation of adipose tissue mass. Today obesity is a global public health concern that is associated with undesired health outcomes and morbidities. Desired treatments for patients with obesity strive to reduce excess body weight, improve obesity-related co-morbidities, and maintain long-term weight reduction. Available treatments for obesity are particularly unsatisfactory for patients with severe obesity. Successful treatment of obesity is associated with alleviation or prevention of T2D. There is a need for alternative treatment options to induce therapeutic weight loss in patients in need of such treatment. Compounds having a ratio that is about 2.5:1 to about 10:1 GIP receptor agonist potency to GLP-1 receptor agonist potency are useful for significantly improving weight management, and can prevent the manifestation of T2D in formerly obese patients susceptible to T2D.

WO2016/111971 describes peptides stated to have GLP-1 and GIP activity. WO2013/164483 also discloses compounds stated to have GLP-1 and GIP activity. US9474780 generally describes compositions containing a GIP/GLP1 co-agonist, administered by parenteral routes, and generally discloses a wide dosage range up to about 30 mg per person per week. US9474780 discloses the use of GIP/GLP1 co-agonists for treating diabetes, obesity, and other conditions. US9474780 describes and claims tirzepatide.

The present invention provides novel dosing regimens of a GIP: GLP-1 Peptide for use in the aforementioned therapies (glycemic control/diabetes, obesity) that include one or more titration doses and a maintenance dose. More specifically, the present invention provides novel dosing regimens that include a titration dose and a maintenance dose wherein the titration dose is about 50% of the maintenance dose and is administered about once weekly for a minimum of about 2 weeks before administration of the maintenance dose. In another aspect, the dosing regimen comprises three titration doses: the first being about 25% of the maintenance dose, the second being about 50% of the maintenance dose and the third being about 75% of the maintenance dose, and a maintenance dose wherein each titration dose is administered about once weekly for a minimum of about 2 weeks before the administration of the next higher dose. In yet a third embodiment, the dosing regimen comprises five titration doses: the first being about 17% of the maintenance dose, the second being about 33% of the maintenance dose, the third being about 50% of the maintenance dose, the fourth being about 66% of the maintenance dose and the fifth being about 83% of the maintenance dose wherein each titration dose is administered about once weekly for a minimum of about 2 weeks before the administration of the next higher dose. Further embodiments are dosing regimens as above where each titration dose is administered about once weekly for about 4 weeks before the administration of the next higher dose begins.

In an embodiment, GIP: GLP-1 Peptides of the present invention have a receptor agonist potency ratio as measured after a 60 minute incubation using a casein cAMP assay normalized against GIP and GLP-1 of about 2.5:1 to about 10:1 GIP to GLP-1. In an embodiment, GIP: GLP-1 Peptides of the present invention have a receptor agonist potency ratio as measured after a 60 minute incubation using a casein cAMP assay normalized against GIP and GLP-1 of about 2.5:1 to about 5:1 GIP to GLP-1. In an embodiment, GIP: GLP-1 Peptides of the present invention have a receptor agonist potency ratio as measured after a 60 minute incubation using a casein cAMP assay normalized against GIP and GLP-1 of about 2.5:1 to about 3.5:1 GIP to GLP-1. In an embodiment, GIP: GLP-1 Peptides of the present invention have a receptor agonist potency ratio as measured after a 60 minute incubation using a casein cAMP assay normalized against GIP and GLP-1 of about 2.5:1 to about 10:1 GIP to GLP-1. In an embodiment, GIP: GLP-1 Peptides of the present invention have a receptor agonist potency ratio as measured after a 60 minute incubation at 37C using a casein cAMP assay normalized against GIP and GLP-1 of about 2.5:1 to about 10:1 GIP to GLP-1.

Accordingly, the present invention provides a method of treating type 2 diabetes in a patient in need thereof, comprising: administering a titration dose of a GIP: GLP-1 Peptide for about two weeks and thereafter administering a maintenance dose of that GIP: GLP-1 Peptide wherein the titration dose is about 50% of the maintenance dose and wherein the GIP: GLP-1 Peptide has a receptor agonist potency ratio as measured after a 60 minute incubation using a casein cAMP assay normalized against GIP and GLP-1 that is about 2.5:1 to about 10:1 GIP to GLP-1.

The present invention further provides a method of improving glycemic control in a patient in need thereof, comprising: administering a titration dose of a GIP: GLP-1 Peptide for about two weeks and thereafter administering a maintenance dose of that GIP: GLP-1 Peptide wherein the titration dose is about 50% of the maintenance dose and wherein the GIP: GLP-1 Peptide has a receptor agonist potency ratio as measured after a 60 minute incubation using a casein cAMP assay normalized against GIP and GLP-1 that is about 2.5:1 to about 10:1 GIP to GLP-1.

Furthermore, the present invention provides a method of improving weight management in a patient in need thereof, comprising: administering a titration dose of a GIP: GLP-1 Peptide for about two weeks and thereafter administering a maintenance dose of that GIP: GLP-1 Peptide wherein the titration dose is about 50% of the maintenance dose and wherein the GIP: GLP-1 Peptide has a receptor agonist potency ratio as measured after a 60 minute incubation using a casein cAMP assay normalized against GIP and GLP-1 that is about 2.5:1 to about 10:1 GIP to GLP-1.

Furthermore, the present invention provides a method of treating chronic kidney disease in a patient in need thereof, comprising: administering a titration dose of a GIP: GLP-1 Peptide for about two weeks and thereafter administering a maintenance dose of that GIP: GLP-1 Peptide wherein the titration dose is about 50% of the maintenance dose and wherein the GIP: GLP-1 Peptide has a receptor agonist potency ratio as measured after a 60 minute incubation using a casein cAMP assay normalized against GIP and GLP-1 that is about 2.5:1 to about 10:1 GIP to GLP-1.

Furthermore, the present invention provides a method of treating atherosclerosis in a patient in need thereof, comprising: administering a titration dose of a GIP: GLP-1 Peptide for about two weeks and thereafter administering a maintenance dose of that GIP: GLP-1 Peptide wherein the titration dose is about 50% of the maintenance dose and wherein the GIP: GLP-1 Peptide has a receptor agonist potency ratio as measured after a 60 minute incubation using a casein cAMP assay normalized against GIP and GLP-1 that is about 2.5:1 to about 10:1 GIP to GLP-1.

Furthermore, the present invention provides a method of treating NAFLD in a patient in need thereof, comprising: administering a titration dose of a GIP: GLP-1 Peptide for about two weeks and thereafter administering a maintenance dose of that GIP: GLP-1 Peptide wherein the titration dose is about 50% of the maintenance dose and wherein the GIP: GLP-1 Peptide has a receptor agonist potency ratio as measured after a 60 minute incubation using a casein cAMP assay normalized against GIP and GLP-1 that is about 2.5:1 to about 10:1 GIP to GLP-1.

Furthermore, the present invention provides a method of treating NASH in a patient in need thereof, comprising: administering a titration dose of a GIP: GLP-1 Peptide for about two weeks and thereafter administering a maintenance dose of that GIP: GLP-1 Peptide wherein the titration dose is about 50% of the maintenance dose and wherein the GIP: GLP-1 Peptide has a receptor agonist potency ratio as measured after a 60 minute incubation using a casein cAMP assay normalized against GIP and GLP-1 that is about 2.5:1 to about 10:1 GIP to GLP-1.

Furthermore, the present invention provides a method of curing diabetes, inducing remission or regression of diabetes or preventing diabetes in a patient in need thereof, comprising: administering a titration dose of a GIP: GLP-1 Peptide for about two weeks and thereafter administering a maintenance dose of that GIP: GLP-1 Peptide wherein the titration dose is about 50% of the maintenance dose and wherein the GIP: GLP-1 Peptide has a receptor agonist potency ratio as measured after a 60 minute incubation using a casein cAMP assay normalized against GIP and GLP-1 that is about 2.5:1 to about 10:1 GIP to GLP-1.

A further embodiment of the present invention are the methods above wherein three titration doses (about 25%, about 50% and about 75% of the maintenance dose) are administered starting with the 25% dose and wherein each titration dose is administered for about two weeks before the administration of the next higher dose begins. A further embodiment of the present invention are the methods above wherein five titration doses (about 17%, about 33%, about 50%, about 66% and about 83% of the maintenance dose) are administered starting with the 17% dose and wherein each titration dose is administered for about two weeks before the administration of the next higher dose. A further embodiment of the present invention are the methods above where the titration dose or doses are administered for about four weeks before the administration of the next higher dose begins.

Another aspect of the present invention are methods described above for treating type 2 diabetes, improving glycemic control, improving weight management, treating chronic kidney disease, treating NAFLD, treating NASH and to cure diabetes, induce remission or regression of diabetes, or prevent diabetes wherein the GIP: GLP-1 Peptide has a GIP: GLP-1 receptor agonist potency ratio that is about 2.5:1 to about 5:1 as measured after a 60 minute incubation using a casein cAMP assay normalized against GIP and GLP-1. A further aspect of the present invention are methods described above for treating type 2 diabetes, improving glycemic control, improving weight management, treating chronic kidney disease, treating NAFLD, treating NASH or to cure diabetes, induce remission or regression of diabetes, or prevent diabetes wherein the GIP: GLP-1 Peptide has a GIP: GLP-1 receptor agonist potency ratio that is about 2.5:1 to about 3.5:1 as measured after a 60 minute incubation using a casein cAMP assay normalized against GIP and GLP-1.

A further aspect of the present invention are the methods described above for treating type 2 diabetes, improving glycemic control, improving weight management, treating chronic kidney disease, treating NAFLD, treating NASH or to cure diabetes, induce remission or regression of diabetes, or prevent diabetes wherein the GIP: GLP-1 Peptide is a compound of SEQ ID NO: 3, or a pharmaceutically acceptable salt thereof. Another aspect of the present invention are the methods described above for treating type 2 diabetes, improving glycemic control, improving weight management, treating chronic kidney disease, treating NAFLD, treating NASH or to cure diabetes, induce remission or regression of diabetes, or prevent diabetes wherein the GIP: GLP-1 Peptide is a compound of SEQ ID NO: 3, or a pharmaceutically acceptable salt thereof, and wherein the compound of SEQ ID NO: 3, or a pharmaceutically acceptable salt thereof, has a GIP: GLP-1 receptor agonist potency ratio that is about 2.5:1 to about 5:1 as measured after a 60 minute incubation using a casein cAMP assay normalized against GIP and GLP-1. Another aspect of the present invention are the methods described above for treating type 2 diabetes, improving glycemic control, improving weight management, treating chronic kidney disease, treating NAFLD, treating NASH or to cure diabetes, induce remission or regression of diabetes, or prevent diabetes wherein the GIP: GLP-1 Peptide is a compound of SEQ ID NO: 3, or a pharmaceutically acceptable salt thereof, and wherein the compound of SEQ ID NO: 3, or a pharmaceutically acceptable salt thereof, has a GIP: GLP-1 receptor agonist potency ratio that is about 2.5:1 to about 3.5:1 as measured after a 60 minute incubation using a casein cAMP assay normalized against GIP and GLP-1.

In an embodiment 1 (a), the present invention provides a method of treating type 2 diabetes in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide; wherein the titration dose is about 50% of the maintenance dose.

In embodiment 1 (b), the present invention provides a method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In embodiment 1 (c), the present invention provides a method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 1 (a1), the present invention provides a method of treating type 2 diabetes in a patient in need thereof, comprising:
a) subcutaneously administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 about once weekly for a minimum of about two weeks; and thereafter
b) administering to said patient a maintenance dose of that GIP: GLP-1 Peptide;
   wherein the titration dose is about 50% of the maintenance dose.

In embodiment 1 (b1), the present invention provides a method of treating type 2 diabetes in a patient in need thereof, comprising:
a) subcutaneously administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 about once weekly for a minimum of about two weeks; and thereafter
b) subcutaneously administering to said patient a second titration dose of that GIP: GLP-1 Peptide about once weekly for a minimum of about two weeks; and thereafter
c) subcutaneously administering to said patient a third titration dose of that GIP: GLP-1 Peptide about once weekly for a minimum of about two weeks; and thereafter
d) subcutaneously administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In embodiment 1 (c1), the present invention provides a method of treating type 2 diabetes in a patient in need thereof, comprising:
a) subcutaneously administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 about once weekly for a minimum of about two weeks; and thereafter
b) subcutaneously administering to said patient a second titration dose of that GIP: GLP-1 Peptide about once weekly for a minimum of about two weeks; and thereafter
c) subcutaneously administering to said patient a third titration dose of that GIP: GLP-1 Peptide about once weekly for a minimum of about two weeks; and thereafter
d) subcutaneously administering to said patient a fourth titration dose of that GIP: GLP-1 Peptide about once weekly for a minimum of about two weeks; and thereafter
e) subcutaneously administering to said patient a fifth titration dose of that GIP: GLP-1 Peptide about once weekly for a minimum of about two weeks; and thereafter
f) subcutaneously administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 2 (a), the present invention provides a method of improving glycemic control in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In an embodiment 2 (a), the present invention provides a method of improving glycemic control in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In embodiment 2 (b), the present invention provides a method of improving glycemic control in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In embodiment 2 (c), the present invention provides a method of improving glycemic control in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 3 (a), the present invention provides a method of improving weight management in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In embodiment 3 (b), the present invention provides a method of improving weight management in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In embodiment 3 (c), the present invention provides a method of improving weight management in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 4 (a), the present invention provides a method of treating chronic kidney disease in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In embodiment 4 (b), the present invention provides a method of treating chronic kidney disease in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In embodiment 4 (c), the present invention provides a method of treating chronic kidney disease in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 5 (a), the present invention provides a method of treating NAFLD in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In embodiment 5 (b), the present invention provides a method of treating NAFLD in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In embodiment 5 (c), the present invention provides a method of treating NAFLD in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 6 (a), the present invention provides a method of treating NASH in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In embodiment 6 (b), the present invention provides a method of treating NASH in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In embodiment 6 (c), the present invention provides a method of treating NASH in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 7 (a), the present invention provides a method of curing diabetes, inducing remission or regression of diabetes, or preventing diabetes in a patient in need thereof, comprising: to cure diabetes, induce remission or regression of diabetes, or prevent diabetes
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In embodiment 7 (b), the present invention provides a method of curing diabetes, inducing remission or regression of diabetes, or preventing diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In embodiment 7 (c), the present invention provides a method of curing diabetes, inducing remission or regression of diabetes, or preventing diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In embodiment 8, the titration doses of embodiments 1 (a)-(c), 2 (a)-(c), 3 (a)-(c), 4 (a)-(c), 5 (a)-(c), 6 (a)-(c) or 7 (a)-(c) are each administered for about four weeks before the administration of the next higher dose begins. Thus, for example, the present invention provides in embodiment 1 (d) a method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for about four weeks; and thereafter
b) administering to said patient a maintenance dose of that GIP: GLP-1 Peptide;
   wherein the titration dose is about 50% of the maintenance dose.

The present invention thus further provides in embodiment 1 (e), a method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for about four weeks; and thereafter
b) administering to said patient a second titration dose of that GIP: GLP-1 Peptide for about four weeks; and thereafter
c) administering to said patient a third titration dose of that GIP: GLP-1 Peptide for about four weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

The present invention thus further provides in embodiment 1 (f), a method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for about four weeks; and thereafter
b) administering to said patient a second titration dose of that GIP: GLP-1 Peptide for about four weeks; and thereafter
c) administering to said patient a third titration dose of that GIP: GLP-1 Peptide for about four weeks; and thereafter
d) administering to said patient a fourth titration dose of that GIP: GLP-1 Peptide for about four weeks; and thereafter
e) administering to said patient a fifth titration dose of that GIP: GLP-1 Peptide for about four weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In embodiment 9, the GIP: GLP-1 Peptide of embodiments 1 (a)-(c), 2 (a)-(c), 3 (a)-(c), 4 (a)-(c), 5 (a)-(c), 6 (a)-(c), 7 (a)-(c) or 8 is a compound of SEQ ID NO: 3, or a pharmaceutically acceptable salt thereof. Thus, for example, the present invention provides in embodiment 1 (g) a method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 wherein the GIP: GLP-1 Peptide is a compound of SEQ ID NO: 3 for about four weeks; and thereafter
b) administering to said patient a maintenance dose of that GIP: GLP-1 Peptide;
   wherein the titration dose is about 50% of the maintenance dose.

The present invention thus further provides in embodiment 1 (h), a method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 wherein the GIP: GLP-1 Peptide is a compound of SEQ ID NO: 3 for about four weeks; and thereafter
b) administering to said patient a second titration dose of that GIP: GLP-1 Peptide for about four weeks; and thereafter
c) administering to said patient a third titration dose of that GIP: GLP-1 Peptide for about four weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

The present invention thus further provides in embodiment 1 (i), a method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 wherein the GIP: GLP-1 Peptide is a compound of SEQ ID NO: 3 for about four weeks; and thereafter
b) administering to said patient a second titration dose of that GIP: GLP-1 Peptide for about four weeks; and thereafter
c) administering to said patient a third titration dose of that GIP: GLP-1 Peptide for about four weeks; and thereafter
d) administering to said patient a fourth titration dose of that GIP: GLP-1 Peptide for about four weeks; and thereafter
e) administering to said patient a fifth titration dose of that GIP: GLP-1 Peptide for about four weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In embodiment 10, the GIP: GLP-1 Peptide of embodiments 1 (a)-(c), 2 (a)-(c), 3 (a)-(c), 4 (a)-(c), 5 (a)-(c), 6 (a)-(c), 7 (a)-(c), 8 or 9 has a receptor agonist potency ratio that is 2.5:1 to about 5:1 GIP to GLP-1.

In embodiment 11, the GIP: GLP-1 Peptide of embodiments 1 (a)-(c), 2 (a)-(c), 3 (a)-(c), 4 (a)-(c), 5 (a)-(c), 6 (a)-(c), 7 (a)-(c), 8, 9 or 10 has a receptor agonist potency ratio that is 2.5:1 to about 3.5:1 GIP to GLP-1.

In an embodiment 12 (a), the present invention provides the use of a GIP: GLP-1 Peptide, or a pharmaceutically acceptable salt thereof, in the manufacture of a medicament for treating type 2 diabetes in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In an embodiment 12 (b), the present invention provides the use of a GIP: GLP-1 Peptide, or a pharmaceutically acceptable salt thereof, in the manufacture of a medicament for treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In an embodiment 12 (c), the present invention provides the use of a GIP: GLP-1 Peptide, or a pharmaceutically acceptable salt thereof, in the manufacture of a medicament for treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 13 (a), the present invention provides the use of a GIP: GLP-1 Peptide, or a pharmaceutically acceptable salt thereof, in the manufacture of a medicament for improving glycemic control in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In an embodiment 13 (b), the present invention provides the use of a GIP: GLP-1 Peptide, or a pharmaceutically acceptable salt thereof, in the manufacture of a medicament for improving glycemic control in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In an embodiment 13 (c), the present invention provides the use of a GIP: GLP-1 Peptide, or a pharmaceutically acceptable salt thereof, in the manufacture of a medicament for improving glycemic control in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP; GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 14 (a), the present invention provides the use of a GIP: GLP-1 Peptide, or a pharmaceutically acceptable salt thereof, in the manufacture of a medicament for improving weight management in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In an embodiment 14 (b), the present invention provides the use of a GIP: GLP-1 Peptide, or a pharmaceutically acceptable salt thereof, in the manufacture of a medicament for improving weight management in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In an embodiment 14 (c), the present invention provides the use of a GIP: GLP-1 Peptide, or a pharmaceutically acceptable salt thereof, in the manufacture of a medicament for improving weight management in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 15 (a), the present invention provides the use of a GIP: GLP-1 Peptide, or a pharmaceutically acceptable salt thereof, in the manufacture of a medicament for treating chronic kidney disease in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In an embodiment 15 (b), the present invention provides the use of a GIP: GLP-1 Peptide, or a pharmaceutically acceptable salt thereof, in the manufacture of a medicament for treating chronic kidney disease in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In an embodiment 15 (c), the present invention provides the use of a GIP: GLP-1 Peptide, or a pharmaceutically acceptable salt thereof, in the manufacture of a medicament for treating chronic kidney disease in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 16 (a), the present invention provides the use of a GIP: GLP-1 Peptide, or a pharmaceutically acceptable salt thereof, in the manufacture of a medicament for treating NAFLD in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In an embodiment 16 (b), the present invention provides the use of a GIP: GLP-1 Peptide, or a pharmaceutically acceptable salt thereof, in the manufacture of a medicament for treating NAFLD in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In an embodiment 16 (c), the present invention provides the use of a GIP: GLP-1 Peptide, or a pharmaceutically acceptable salt thereof, in the manufacture of a medicament for treating NAFLD in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 17 (a), the present invention provides the use of a GIP: GLP-1 Peptide, or a pharmaceutically acceptable salt thereof, in the manufacture of a medicament for treating NASH in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In an embodiment 17 (b), the present invention provides the use of a GIP: GLP-1 Peptide, or a pharmaceutically acceptable salt thereof, in the manufacture of a medicament for treating NASH in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In an embodiment 17 (c), the present invention provides the use of a GIP: GLP-1 Peptide, or a pharmaceutically acceptable salt thereof, in the manufacture of a medicament for treating NASH in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 18 (a), the present invention provides the use of a GIP: GLP-1 Peptide, or a pharmaceutically acceptable salt thereof, in the manufacture of a medicament for inducing remission or regression of diabetes, or preventing diabetes in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
   wherein the titration dose is about 50% of the maintenance dose.

In an embodiment 18 (b), the present invention provides the use of a GIP: GLP-1 Peptide, or a pharmaceutically acceptable salt thereof, in the manufacture of a medicament for inducing remission or regression of diabetes, or preventing diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
   wherein the first titration dose is about 25% of the maintenance dose, the second titration dose is about 50% of the maintenance dose and the third titration dose is about 75% of the maintenance dose.

In an embodiment 18 (c), the present invention provides the use of a GIP: GLP-1 Peptide, or a pharmaceutically acceptable salt thereof, in the manufacture of a medicament for inducing remission or regression of diabetes, or preventing diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In embodiment 19, the titration doses of any of embodiments 12 (a)-(c), 13 (a)-(c), 14 (a)-(c), 15 (a)-(c), 16 (a)-(c), 17 (a)-(c), or 18 (a)-(c) are each administered for about four weeks before the administration of the next higher dose begins.

In embodiment 20, the GIP: GLP-1 Peptide of any of embodiments 12 (a)-(c), 13 (a)-(c), 14 (a)-(c), 15 (a)-(c), 16 (a)-(c), 17 (a)-(c), 18 (a)-(c), or 19 is a peptide of SEQ ID NO: 3, or a pharmaceutically acceptable salt thereof.

In embodiment 21, the GIP: GLP-1 Peptide of any of embodiments 12 (a)-(c), 13 (a)-(c), 14 (a)-(c), 15 (a)-(c), 16 (a)-(c), 17 (a)-(c), 18 (a)-(c), 19 or 20 has a receptor agonist potency ratio that is 2.5:1 to about 5:1 GIP to GLP-1.

In embodiment 22, the GIP: GLP-1 Peptide of any of embodiments 12 (a)-(c), 13 (a)-(c), 14 (a)-(c), 15 (a)-(c), 16 (a)-(c), 17 (a)-(c), 18 (a)-(c), 19, or 20 has a receptor agonist potency ratio that is 2.5:1 to about 3.5:1 GIP to GLP-1.

In an embodiment 23 (a), the present invention provides a GIP: GLP-1 Peptide, or a pharmaceutically acceptable salt thereof, for use in treating type 2 diabetes in a patient in need thereof wherein the use comprises:
a) administration of a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a maintenance dose of that GIP: GLP-1 Peptide;
wherein the titration dose is about 50% of the maintenance dose.

In an embodiment 23 (b), the present invention provides a GIP: GLP-1 Peptide, or a pharmaceutically acceptable salt thereof, for use in treating type 2 diabetes in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a maintenance dose of that GIP: GLP-1 Peptide;
wherein the first titration dose is about 25% of the maintenance dose, the second titration dose is about 50% of the maintenance dose and the third titration dose is about 75% of the maintenance dose.

In an embodiment 23 (c), the present invention provides a GIP: GLP-1 Peptide, or a pharmaceutically acceptable salt thereof, for use in treating type 2 diabetes in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a fourth titration dose of that GIP: GLP-1 Peptide for a minimum of about two weeks; and thereafter
e) administration of a fifth titration dose of that GIP: GLP-1 Peptide for a minimum of about two weeks; and thereafter
f) administration of a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 24 (a), the present invention provides a GIP: GLP-1 Peptide, or a pharmaceutically acceptable salt thereof, for use in improving glycemic control in a
patient in need thereof wherein the use comprises: a) administration of a titration dose of a GIP: GLP-1 Peptide having a

GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a maintenance dose of that GIP: GLP-1 Peptide;
wherein the titration dose is about 50% of the maintenance dose.

In an embodiment 24 (b), the present invention provides a GIP: GLP-1 Peptide, or a pharmaceutically acceptable salt thereof, for use in improving glycemic control in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a maintenance dose of that GIP: GLP-1 Peptide;
wherein the first titration dose is about 25% of the maintenance dose, the second titration dose is about 50% of the maintenance dose and the third titration dose is about 75% of the maintenance dose.

In an embodiment 24 (c), the present invention provides a GIP: GLP-1 Peptide, or a pharmaceutically acceptable salt thereof, for use in improving glycemic control in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a fourth titration dose of that GIP: GLP-1 Peptide for a minimum of about two weeks; and thereafter
e) administration of a fifth titration dose of that GIP: GLP-1 Peptide for a minimum of about two weeks; and thereafter
f) administration of a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 25 (a), the present invention provides a GIP: GLP-1 Peptide, or a pharmaceutically acceptable salt thereof, for use in improving weight management in a patient in need thereof wherein the use comprises:
a) administration of a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a maintenance dose of that GIP: GLP-1 Peptide;
wherein the titration dose is about 50% of the maintenance dose.

In an embodiment 25 (b), the present invention provides a GIP: GLP-1 Peptide, or a pharmaceutically acceptable salt thereof, for use in improving weight management in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a maintenance dose of that GIP: GLP-1 Peptide;
wherein the first titration dose is about 25% of the maintenance dose, the second titration dose is about 50% of the maintenance dose and the third titration dose is about 75% of the maintenance dose.

In an embodiment 25 (c), the present invention provides a GIP: GLP-1 Peptide, or a pharmaceutically acceptable salt thereof, for use in improving weight management in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a fourth titration dose of that GIP: GLP-1 Peptide for a minimum of about two weeks; and thereafter
e) administration of a fifth titration dose of that GIP: GLP-1 Peptide for a minimum of about two weeks; and thereafter
f) administration of a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 26 (a), the present invention provides a GIP: GLP-1 Peptide, or a pharmaceutically acceptable salt thereof, for use in treating chronic kidney disease in a patient in need thereof wherein the use comprises:
a) administration of a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a maintenance dose of that GIP: GLP-1 Peptide;
wherein the titration dose is about 50% of the maintenance dose.

In an embodiment 26 (b), the present invention provides a GIP: GLP-1 Peptide, or a pharmaceutically acceptable salt thereof, for use in treating chronic kidney disease in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a maintenance dose of that GIP: GLP-1 Peptide;
wherein the first titration dose is about 25% of the maintenance dose, the second titration dose is about 50% of the maintenance dose and the third titration dose is about 75% of the maintenance dose.

In an embodiment 26 (c), the present invention provides a GIP: GLP-1 Peptide, or a pharmaceutically acceptable salt thereof, for use in treating chronic kidney disease in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a fourth titration dose of that GIP: GLP-1 Peptide for a minimum of about two weeks; and thereafter
e) administration of a fifth titration dose of that GIP: GLP-1 Peptide for a minimum of about two weeks; and thereafter
f) administration of a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 27 (a), the present invention provides a GIP: GLP-1 Peptide, or a pharmaceutically acceptable salt thereof, for use in treating NAFLD in a patient in need thereof wherein the use comprises:
a) administration of a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a maintenance dose of that GIP: GLP-1 Peptide;
wherein the titration dose is about 50% of the maintenance dose.

In an embodiment 27 (b), the present invention provides a GIP: GLP-1 Peptide, or a pharmaceutically acceptable salt thereof, for use in treating NAFLD in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a maintenance dose of that GIP: GLP-1 Peptide;
wherein the first titration dose is about 25% of the maintenance dose, the second titration dose is about 50% of the maintenance dose and the third titration dose is about 75% of the maintenance dose.

In an embodiment 27 (c), the present invention provides a GIP: GLP-1 Peptide, or a pharmaceutically acceptable salt thereof, for use in treating NAFLD in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a fourth titration dose of that GIP: GLP-1 Peptide for a minimum of about two weeks; and thereafter
e) administration of a fifth titration dose of that GIP: GLP-1 Peptide for a minimum of about two weeks; and thereafter
f) administration of a maintenance dose of that GIP: GLP-1 Peptide;
   wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 28 (a), the present invention provides a GIP: GLP-1 Peptide, or a pharmaceutically acceptable salt thereof, for use in treating NASH in a patient in need thereof wherein the use comprises:
a) administration of a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a maintenance dose of that GIP: GLP-1 Peptide;
wherein the titration dose is about 50% of the maintenance dose.

In an embodiment 28 (b), the present invention provides a GIP: GLP-1 Peptide, or a pharmaceutically acceptable salt thereof, for use in treating NASH in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a maintenance dose of that GIP: GLP-1 Peptide;
wherein the first titration dose is about 25% of the maintenance dose, the second titration dose is about 50% of the maintenance dose and the third titration dose is about 75% of the maintenance dose.

In an embodiment 28 (c), the present invention provides a GIP: GLP-1 Peptide, or a pharmaceutically acceptable salt thereof, for use in treating NASH in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a fourth titration dose of that GIP: GLP-1 Peptide for a minimum of about two weeks; and thereafter
e) administration of a fifth titration dose of that GIP: GLP-1 Peptide for a minimum of about two weeks; and thereafter
f) administration of a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In an embodiment 29 (a), the present invention provides a GIP: GLP-1 Peptide, or a pharmaceutically acceptable salt thereof, for use in inducing remission or regression of diabetes, or preventing diabetes in a patient in need thereof wherein the use comprises:
a) administration of a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a maintenance dose of that GIP: GLP-1 Peptide;
wherein the titration dose is about 50% of the maintenance dose.

In an embodiment 29 (b), the present invention provides a GIP: GLP-1 Peptide, or a pharmaceutically acceptable salt thereof, for use in inducing remission or regression of diabetes, or preventing diabetes in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a maintenance dose of that GIP: GLP-1 Peptide;
wherein the first titration dose is about 25% of the maintenance dose, the second titration dose is about 50% of the maintenance dose and the third titration dose is about 75% of the maintenance dose.

In an embodiment 29 (c), the present invention provides a GIP: GLP-1 Peptide, or a pharmaceutically acceptable salt thereof, for use in inducing remission or regression of diabetes, or preventing diabetes in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a fourth titration dose of that GIP: GLP-1 Peptide for a minimum of about two weeks; and thereafter
e) administration of a fifth titration dose of that GIP: GLP-1 Peptide for a minimum of about two weeks; and thereafter
f) administration of a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

In embodiment 30, the titration doses of any of embodiments 23 (a)-(c), 24 (a)-(c), 25 (a)-(c), 26 (a)-(c), 27 (a)-(c), 28 (a)-(c), or 29 (a)-(c) are each administered for about four weeks before the administration of the next higher dose begins,

In embodiment 31, the GIP: GLP-1 Peptide of any of embodiments 23 (a)-(c), 24 (a)-(c), 25 (a)-(c), 26 (a)-(c), 27 (a)-(c), 28 (a)-(c), 29 (a)-(c) or 30 is a peptide of SEQ ID NO: 3, or a pharmaceutically acceptable salt thereof.

In embodiment 32, the GIP: GLP-1 Peptide of any of embodiments 23 (a)-(c), 24 (a)-(c), 25 (a)-(c), 26 (a)-(c), 27 (a)-(c), 28 (a)-(c), 29 (a)-(c), 30, or 31 has a receptor agonist potency ratio that is 2.5:1 to about 5:1 GIP to GLP-1.

In embodiment 33, the GIP: GLP-1 Peptide of any of embodiments 23 (a)-(c), 24 (a)-(c), 25 (a)-(c), 26 (a)-(c), 27 (a)-(c), 28 (a)-(c), 29 (a)-(c), 30, or 31 has a receptor agonist potency ratio that is 2.5:1 to about 3.5:1 GIP to GLP-1.

In embodiment 34, is a composition comprising a GIP-GLP-1 Peptide having a GIP: GLP-1 receptor agonist potency ratio as measured after a 60 minute incubation using casein cAMP assay normalized against GIP and GLP-1 that is about 2.5:1 to about 10:1; and a pharmaceutically acceptable excipient.

In embodiment 34 (a) is a composition administered at least two weeks as an escalation dose.

In embodiment 34 (al) is a composition administered at least two weeks as an escalation dose.

In embodiment 34 (b) is a composition administered as a maintenance dose for at least two weeks.

In embodiment 35, is a GIP-GLP-1 Peptide for use to treat diabetes in a patient in need thereof, wherein the GIP: GLP-1 receptor potency ratio of the GIP-GLP-1 Peptide, as measured after a 60 minute incubation using a casein cAMP assay normalized against GIP and GLP-1, is about 2.5:1 to about 10:1.

In embodiment 35 (a) is a GIP-GLP-1 Peptide of embodiment 35 wherein the Peptide is administered for a minimum of about 2 weeks.

In embodiment 35 (b) is a GIP-GLP-1 Peptide of embodiment 35 or 35 (a) wherein the Peptide is administered as at least one escalation dose for a minimum of about 2 weeks.

Also provided herein are methods of using a GIP: GLP-1 Peptide in simultaneous, separate and sequential combinations with one or more agents selected from metformin, a thiazolidinedione, a sulfonylurea, a dipeptidyl peptidase 4 inhibitor, a sodium glucose co-transporter, a SGLT-2 inhibitor, a growth differentiation factor 15 modulator (“GDF15”), a peptide tyrosine tyrosine modulator (“PYY”), a modified insulin, amylin, a dual amylin calcitonin receptor agonist, and oxyntomodulin agonist (“OXM”) in the treatment of a condition selected from the group consisting of type 2 diabetes, chronic kidney disease, atherosclerosis, NALED and NASH. Further provided herein are methods of using a GIP: GLP-1 Peptide in simultaneous, separate and sequential combinations with one or more agents selected from metformin, a thiazolidinedione, a sulfonylurea, a dipeptidyl peptidase 4 inhibitor, a sodium glucose co-transporter, a SGLT-2 inhibitor, GDF15, PYY, a modified insulin, amylin, a dual amylin calcitonin receptor agonist, and OXM in the improvement of glycemic control and/or weight management. Also provided herein are methods of using a GIP: GLP-1 Peptide in simultaneous, separate and sequential combinations with one or more agents selected from metformin, a thiazolidinedione, a sulfonylurea, a dipeptidyl peptidase 4 inhibitor, a sodium glucose co-transporter, a SGLT-2 inhibitor, GDF15, PYY, a modified insulin, amylin, a dual amylin calcitonin receptor agonist, and OXM to cure diabetes, induce remission or regression of diabetes, or prevent diabetes. In an embodiment, a compound of the present invention is provided in a fixed dose combination with one or more agents selected from metformin, a thiazolidinedione, a sulfonylurea, a dipeptidyl peptidase 4 inhibitor, a sodium glucose co-transporter, a SGLT-2 inhibitor, GDF15, PYY, a modified insulin, amylin, a dual amylin calcitonin receptor agonist, and OXM.

The present invention provides novel dosing regimens that include administering a titration dose about once weekly for a minimum of about two weeks and thereafter administering a maintenance dose wherein the titration dose is about 50% of the maintenance dose. In certain embodiment, the titration dose may be administered for about four weeks. In certain embodiments, the titration dose may be administered for more than about four weeks as determined by the nurse, patient and/or health care provider.

As used herein, the term “treating” or “to treat” includes restraining, slowing, stopping, or reversing the progression or severity of a symptom, condition, or disorder.

As used herein, “normalized against GIP and GLP-1” means that the native peptides, as provided herein as SEQ ID NO:1 and SEQ ID NO:2, are tested in the casein cAMP assay as a control for the test compound, and Raw CPM data for concentration curves of peptides, GLP-1, or GIP are converted to percent inhibition by subtracting nonspecific binding (binding in the presence of excess unlabeled GLP-1, or GIP, respectively) from the individual CPM values and dividing by the total binding signal, also corrected by subtracting nonspecific binding. Data are analyzed using four-parameter (curve maximum, curve minimum, IC₅₀, Hill slope) nonlinear regression routines (Genedata Screener, version 12.0.4, Genedata AG, Basal, Switzerland).

GIP is a 42 amino acid peptide (SEQ ID NO:1), which, like GLP-1, is also known as an incretin, and plays a physiological role in glucose homeostasis by stimulating insulin secretion from pancreatic beta cells in the presence of glucose. GLP-1 is a 36 amino acid peptide, the major biologically active fragment of which (GLP-17-36) is produced as a 30-amino acid, C-terminal amidated peptide (SEQ ID NO:2).

The compounds of SEQ ID NO: 3 provide desired potency at each of the GIP and GLP-1 receptors. In an embodiment, compounds of SEQ ID NO: 3 have desirable GIP and GLP receptor activity wherein the GIP agonist potency is from about 2.5:1 to about 10 times the GLP1 receptor potency as measured by the casein cAMP assay, wherein the potency is normalized against native GIP and GLP on the day the assay is run. In an embodiment, compounds of SEQ ID NO: 3 have desirable GIP and GLP receptor activity wherein the GIP agonist potency is from about 2.5:1 to about 5 times the GLP1 receptor potency as measured by the casein cAMP assay, described herein below, wherein the potency is normalized against native GIP and GLP on the day the assay is run.

As used herein “maintenance dose” means an effective dose to treat the patient with a side-effect profile that supports chronic dosing. The term “effective dose” refers to the amount or dose of a GIP: GLP-1 Peptide, which, upon single or multiple dose administration to the patient, provides the desired effect in the patient under diagnosis or treatment. An effective doe can be determined by a person of skill in the art using the clinical trial descriptions set forth herein together with known techniques and by observing results obtained under analogous circumstances. In determining the effective amount for a subject, a number of factors are considered, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

As used herein, the term “titration” or “titration dose(s)” also means and can be interchangable with an escalation or escalation dose(s).

As used herein, the term GIP to GLP-1 also means and can be interchangeable with GIP: GLP-1. Thus, when “about 2.5 to about 10:1 GIP to GLP-1” or “about 2.5:1 to about 10:1 GIP to GLP-1” is used, it also means about 2.5 to about 10 GIP activity to 1 GLP-1 activity or about 2.5 to about 10 GIP activity: 1 GLP-1 activity.

When used herein in reference to one or more of the GIP or GLP-1 receptors, the terms “activity,” “activate[s]” “activat[ing]” and the like refers to the capacity of a compound to bind to and induce a response at the receptor(s), as measured using assays known in the art, such as the in vitro assays described below. Such activity may be measured in vivo using serum. The receptor agonist potency ratio of a GIP: GLP-1 Peptide is measured using the casein cAMP assay, as described herein below.

The affinity of a particular GIP: GLP-1 co-agonist compound for each of the GIP and GLP-1 receptors may be measured using techniques known for measuring receptor binding levels in the art, including, for example those described in the examples below, and is commonly expressed as a Ki value; however, the GIP potency ratio that is about 2.5 to about 10 times the receptor potency at the GLP-1 receptor determined using the casein cAMP assay, below.

In an embodiment, a pharmaceutical composition of a GIP: GLP-1 Peptide is suitable for administration by a parenteral route (e.g., subcutaneous, intravenous, intraperitoneal, intramuscular, or transdermal). In an embodiment, a pharmaceutical composition of a GIP: GLP-1 Peptide is suitable for oral administration (e.g., tablet, capsule). Such pharmaceutical compositions and processes for preparing same are generally well known in the art. (See, e.g., Remington: The Science and Practice of Pharmacy (D. B. Troy, Editor, 21st Edition, Lippincott, Williams & Wilkins, 2006). In an embodiment of the present invention, the route of administration is subcutaneous. In an embodiment of the present invention, the route of administration is oral.

As used herein “glycemic control” refers to the maintenance or reduction of a patient's HbA1c levels; “improving” glycemic control refers to reductions in HbA1c.

As used herein “weight management” refers to the management of obesity in an individual; “improving” weight management refers to a reduction in body weight.

As used herein “HbA1c” refers to glycated hemoglobin levels, which develop when hemoglobin joins with glucose in the blood, HbA1c levels are a commonly used measure of glycemic control in patients with diabetes, with decreased HbA1c levels generally indicating improved glycemic control. In the context of the methods of the present invention, the methods of the present invention result in a decrease in HbA1c. In certain embodiments, the decrease in HbA1c is decreased relative to the HbA1c levels resulting from treatment with a lower dose of a GIP: GLP-1 Peptide.

As used herein, the term “administering” means the administration by a nurse, health care provider, patient or any other individual including self-administration as directed by the doctor. This includes not only delivering into the body but also prescribing, dispensing or assisting in any way with delivery.

As used herein, the terms “treatment,” “treat,” “treating,” and the like, mean to include slowing or attenuating the progression of a disease or disorder. The terms mean to alleviate, ameliorate, or reduce one or more symptoms of a disorder or condition, even if the disorder or condition is not eliminated. The term includes preventing the manifestation.

As used herein “diabetes remission” means that a patient, using a GIP: GLP-1 Peptide for the treatment of diabetes reaches their glycemic control treatment goal.

As used herein, the term GIP: GLP-1 Peptide is a compound, or a pharmaceutically acceptable salt thereof, with a GIP: GLP agonist potency ratio of from about 2.5:1 to about 10:1. The GIP: GLP-1 Peptide treatment to cure diabetes can prevent, reduce the severity of, or induce remission of diabetes in such patient. In an embodiment, a patient using a GIP: GLP-1 Peptide for treatment of diabetes, reaches their glycemic control treatment goal, and requires no concomitant diabetes medicine to maintain the glycemic control goal. In an embodiment, a patient using a GIP: GLP-1 Peptide in the treatment of diabetes reaches at least their glycemic control treatment goal, and the treatment goal is maintained with cessation of treatment using a GIP: GLP-1 Peptide and all other diabetes medication. In an embodiment, a patient using a GIP: GLP-1 Peptide in the treatment of diabetes reaches at least their glycemic control treatment goal, and the treatment goal is maintained for at least a about a month with cessation of treatment using a GIP: GLP-1 Peptide and all other diabetes medications. In an embodiment, a patient using a GIP: GLP-1 Peptide in the treatment of diabetes reaches at least their glycemic control treatment goal, and the treatment goal is maintained for at least about six months with cessation of treatment using a GIP: GLP-1 Peptide and all other diabetes medications. In an embodiment the patient is unable to reach their glycemic goals prior to a GIP: GLP-1 Peptide treatment. In an embodiment, the patient has failed to reach their glycemic goal using oral diabetes medication. In an embodiment, the patient has failed to reach their glycemic goal using metformin treatment. In an embodiment, the patient glycemic goal is less than about 5.7%.

As used herein “patient” or “patients” refers to a mammal in need of treatment for a condition or disorder. In an embodiment, the patient is a human with a disease or condition that would benefit from treatment with a GIP: GLP-1 Peptide having a GIP: GLP-1 receptor agonist potency ratio of from about 2.5:1 to about 10:1.

As used herein “EDTA” means ethylenediaminetetraacetic acid. As used herein “DMSO” means dimethyl sulfoxide. As used herein “CPM” means counts per minute. As used herein “IBMX” means 3-isobutyl-1-methylxanthine. As used herein “LC/MS” means liquid chromatography/mass spectrometry. As used herein “HTRF” means homogeneous time-resolved fluorescence. As used herein “BSA” mean bovine serum albumin.

### Cure Diabetes, Induce Remission or Regression of Diabetes, or Prevent Diabetes

Despite advances in the treatment of diabetes, many patients receiving such treatment are unable to reach their glycemic control goal or HbA1c goal. This invention provides a cure for diabetes wherein a patient receiving treatment for diabetes using a

GIP: GLP1 Peptide, is able to reach their HbA1c goal, and wherein such patient maintains their HbA1c goal after cessation of GIP: GLP1 Peptide treatment. In an embodiment, the patient receiving GIP: GLP1 Peptide treatment for diabetes maintains their HbA1c goal after cessation of all medications approved for use in the treatment of glycemic control or diabetes. As used herein, the term “diabetes medication,” “diabetes medicine” and the like, means a medication approved by the pertinent regulatory agency for use in the treatment of glycemic control or Type II diabetes. In an embodiment, the HbA1c measurement in the patient treated for diabetes is less than or equal to about 5.9%. In an embodiment, the patient maintains their HbA1c goal level for at least one month without further GIP: GLP1 Peptide administration. In an embodiment, the patient previously treated for diabetes using GIP: GLP1 Peptide maintains their HbA1 goal level for at least one month without administration of further GIP: GLP1 Peptide or any other diabetes medication. In an embodiment, the patient maintains their HbA1c goal level for at least 6 months without administration of further GIP: GLP1 Peptide or any other diabetes medication.

As used herein the term “amino acid” means both naturally occurring amino acids and unnatural amino acids. The amino acids are typically depicted using standard one letter codes (e.g., L=leucine), as well as alpha-methyl substituted residues of natural amino acids (e.g., α-methyl leucine, or αMeL and α-methyl lysine, or αMeK) and certain other unnatural amino acids, such as alpha amino isobutyric acid, or “Aib,” “4Pal,” “Orn,” and the like. The structures of these amino acids appear below:

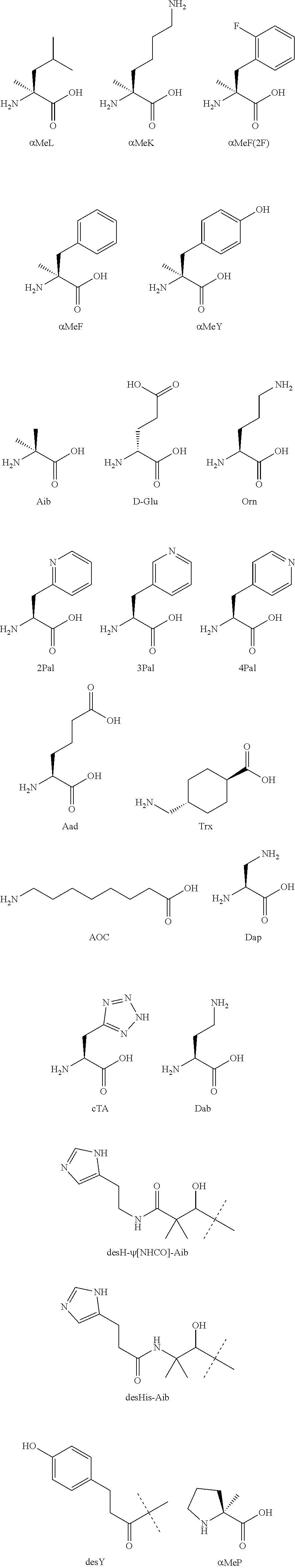

As used herein “Orn” means ornithine. As used herein “4Pal” means 3-(4-Pyridyl)-L-alanine. As used herein “αMeF(2F)” means alpha-methyl 2-Fl-phenylalanine.

As used herein “αMeY,” “αMeK,” and “αMeL” mean alpha methyl tyrosine, alpha methyl lysine, and alpha methyl leucine, respectively. As used herein, “e” and “D-Glu” mean D-glutamic acid.

When used herein, the term “amino acid conjugated to a C₁₆-C₂₂ fatty acid” refers to any natural or unnatural amino acid with a functional group that has been chemically modified to conjugate to a fatty acid by way of a direct bond to the fatty acid or, preferably, by way of a linker. Examples of such functional groups include amino, carboxyl, chloro, bromo, iodo, azido, alkynyl, alkenyl, and thiol groups. Examples of natural amino acids which include such functional groups include K (amino), C (thiol), E (carboxyl) and D (carboxyl). In an embodiment the conjugated amino acid is K.

The term “C₁₆-C₂₂ fatty acid” as used herein means a carboxylic acid with between 16 and 22 carbon atoms. In an embodiment, the C₁₆-C₂₂ fatty acid suitable for use herein can be a saturated diacid. In an embodiment, the fatty acid is C₂₀-C₂₂. In an embodiment q is selected from the group consisting of 14, 16, 18, and 20. In an embodiment q is selected from 18 and 20. In an embodiment q is 18. In an embodiment q is 20.

In an embodiment, specific saturated C₁₆-C₂₂ fatty acids that are suitable for the compounds and uses thereof disclosed herein include, but are not limited to, hexadecanedioic acid (C₁₆ diacid), heptadecanedioic acid (C₁₇ diacid), octadecanedioic acid (C₁₈ diacid), nonadecanedioic acid (C₁₉ diacid), eicosanedioic acid (C₂₀ diacid), heneicosanedioic acid (C₂₁ diacid), docosanedioic acid (C₂₂ diacid), including branched and substituted derivatives thereof.

In an embodiment, the C₁₆-C₂₂ fatty acid is selected from the group consisting of a saturated C₁₈ diacid, a saturated C₁₉ diacid, a saturated C₂₀ diacid, and branched and substituted derivatives thereof. In an embodiment, the C₁₆-C₂₂ fatty acid is selected from the group consisting of stearic acid, arachadic acid and eicosanedioic acid. In an embodiment, the C₁₆-C₂₂ fatty acid is arachadic acid.

As used herein “time-extension technology” means a peptide time-extension technology for example, recombinant human serum albumin (“rHSA”), peptide conjugation to a pharmaceutically acceptable polymer, such as polymeric sequence of amino acids (“XTEN”), unsulfated heparin-like carbohydrate polymer (“HEP”), hydroxyl ethyl starch (“HES”), llama heavy-chain antibody fragments (“VHH”), pegylation, Fc conjugation, bovine serum albumin (“BSA”) (Sleep, D. Epert Opin Drug Del (2015) 12, 793-812; Podust V N et.al. J Control. Release, 2015; ePUB; Hey, T. et. al. in: R. Kontermann (Ed.), Therapeutic Proteins: Strategies to Modulate their Plasma Half-Lives, Wiley-VCH Verlag Gmbh & Co. KGaA, Weinheim, Germany, 2012, pp 117-140; DeAngelis, P L, Drug Dev Delivery (2013) January 12/31/2012. In an embodiment time-extension technology is applied using a linker group. In an embodiment, the time-extension technology is applied using 0, 1, 2, or 3 amino acid as linker.

A compound having a GIP potency ratio that is about 2.5 to about 10 times the receptor potency at the GLP-1 receptor may be further modified using a peptide time-extension technology for example, recombinant human serum albumin (“rHSA”), peptide conjugation to a pharmaceutically acceptable polymer, such as polymeric sequence of amino acids (“XTEN”), unsulfated heparin-like carbohydrate polymer (“HEP”), and hydroxyl ethyl starch (“HES”).

In an embodiment is a GIP: GLP-1 Peptide of the formula:

(SEQ ID NO: 3)
R₁X₁X₂X₃GTX₆TSDX₁₀X₁₁X₁₂X₁₃X₁₄DX₁₆X₁₇AX₁₉X₂₀X₂₁
X₂₂X₂₃X₂₄X₂₅X₂₆X₂₇X₂₈X₂₉X₃₀X₃₁

wherein:
R₁ is a modification of the N-terminal amino group wherein the modification is selected from the group consisting of Ac and absent;
X₁ is selected from the group consisting of Y, H, D-Tyr, F, desH, and desY,
X₂ is selected from the group consisting of Aib, αMeP, A, P, and D-Ala; or X₁ and X₂ combine to form desH-ψ[NHCO]-Aib;
X₃ is selected from the group consisting of E, N, Aad, and cTA;
X₆ is selected from the group consisting of F, αMeF, and αMeF(2F);
X₁₀ is selected from the group consisting of A, L, H, 3Pal, 4Pal, V, Y, E, αMeF, αMeF(2F), I, αMeY, Q, D-His, D-Tyr, cTA, and K (2-[2-(2-amino-ethoxy)-ethoxy]-acetyl)₂-(γ-Glu)-CO—(CH₂)_{q}CO₂H;
X₁₁ is selected from the group consisting of S, αMeS, and D-Ser;
X₁₂ is selected from the group consisting of I, S, D-Ile, and K (2-[2-(2-amino-ethoxy)-ethoxy]-acetyl)₂-(γ-Glu)-CO—(CH₂)_{q}CO₂H;
X₁₃ is selected from the group consisting of Nle, Aib, L, αMeL, and K (2-[2-(2-amino-ethoxy)-ethoxy]-acetyl) 2-(γ-Glu)-CO—(CH₂)_{q}CO₂H;
X₁₄ is selected from the group consisting of L and K, wherein K is conjugated to a C₁₆-C₂₂ fatty acid wherein said fatty acid is optionally conjugated to said K via a linker;
X₁₆ is selected from the group consisting of K, E, Orn, Dab, Dap, S, T, H, Aib, αMeK, R, and K (2-[2-(2-amino-ethoxy)-ethoxy]-acetyl)₂-(γ-Glu)-CO—(CH₂)_{q}CO₂H;
X₁₇ is selected from the group consisting of K, Q, I, and an amino acid conjugated to a C16-C22 fatty acid wherein said fatty acid is optionally conjugated to said amino acid via a linker;
X₁₉ is selected from the group consisting of Q, A, and K (2-[2-(2-amino-ethoxy)-ethoxy]-acetyl)₂-(γ-Glu)-CO—(CH₂)_{q}CO₂H;
X₂₀ is selected from the group consisting of Aib, Q, H, R, K, αMeK, and K (2-[2-(2-amino-ethoxy)-ethoxy]-acetyl)₂-(γ-Glu)-CO—(CH₂)_{q}CO₂H;
X₂₁ is selected from the group consisting of H, Aad, D, Aib, T, A, E, I, and K (2-[2-(2-amino-ethoxy)-ethoxy]-acetyl)₂-(γ-Glu)-CO—(CH₂)_{q}CO₂H;
X₂₂ is selected from the group consisting of F and αMeF;
X₂₃ is selected from the group consisting of I, L, A, G, F, H, E, V, and K (2-[2-(2-amino-ethoxy)-ethoxy]-acetyl)₂-(γ-Glu)-CO—(CH₂)_{q}CO₂H;
X₂₄ is selected from the group consisting of S, Aad, D-Glu, E, Aib, H, V, A, Q, D, P, and K (2-[2-(2-amino-ethoxy)-ethoxy]-acetyl)₂-(Y-Glu)-CO—(CH₂)_{q}CO₂H;
X₂₅ is selected from the group consisting of Y and αMeY;
X₂₆ is selected from the group consisting of L, αMeL, and K (2-[2-(2-amino-ethoxy)-ethoxy]-acetyl)₂-(γ-Glu)-CO—(CH₂)_{q}CO₂H;
X₂₇ is selected from the group consisting of L, I, and K (2-[2-(2-amino-ethoxy)-ethoxy]-acetyl)₂-(γ-Glu)-CO—(CH₂)_{q}CO₂H;
X₂₈ is selected from the group consisting of E, A, S, D-Glu, and K (2-[2-(2-amino-ethoxy)-ethoxy]-acetyl)₂-(γ-Glu)-CO—(CH₂)_{q}CO₂H;
X₂₉ is selected from the group consisting of Aib, G, A, and K (2-[2-(2-amino-ethoxy)-ethoxy]-acetyl), -(γ-Glu)-CO—(CH₂)_{q}CO₂H;
X₃₀ is selected from the group consisting of C, G, G-R₂ and K (2-[2-(2-amino-ethoxy)-ethoxy]-acetyl)₂-(γ-Glu)-CO—(CH₂)_{q}—CO₂H;
X₃₁ is absent or is selected from the group consisting of PX₃₂X₃₃X₃₄—R₂, PX₃₂X₃₃X₃₄X₃₅X₃₆X₃₇X₃₈X₃₉—R₂,PX₃₂X₃₃X₃₄X₃₅X₃₆X₃₇X₃₈X₃₉X₄₀—R₂, K[(2-[2-(2-amino-ethoxy)-ethoxy]-acetyl)₂-(γ-Glu)-CO—(CH₂)_{q}—CO₂H] X₃₂X₃₃X₃₄—R₂, K[(2-[2-(2-amino-ethoxy)-ethoxy]-acetyl)₂-(γ-Glu)-CO—(CH₂)_{q}—CO₂H]
X₃₂X₃₃X₃₄X₃₅X₃₆X₃₇X₃₈X₃₉—R₂, and K[(2-[2-(2-amino-ethoxy)-ethoxy]-acetyl)₂-(γ-Glu)-CO—(CH₂)_{q}—CO₂H] X₃₂X₃₃X₃₄X₃₅X₃₆X₃₇X₃₈X₃₉X₄₀—R₂;
   wherein:
   X₃₂ is S or K[(2-[2-(2-amino-ethoxy)-ethoxy]-acetyl), -(γ-Glu)-CO—(CH₂)_{q}—CO₂H];
   X₃₃ is S or K[(2-[2-(2-amino-ethoxy)-ethoxy]-acetyl), -(γ-Glu)-CO—(CH₂)_{q}—CO₂H];
   X₃₄ is selected from the group consisting of G, C, and K[(2-[2-(2-amino-ethoxy)-ethoxy]-acetyl)₂-(γ-Glu)-CO—(CH₂)_{q}—CO₂H];
   X₃₅ is A or K[(2-[2-(2-amino-ethoxy)-ethoxy]-acetyl), -(γ-Glu)-CO—(CH₂)_{q}—CO₂H];
   X₃₆ is P or K[(2-[2-(2-amino-ethoxy)-ethoxy]-acetyl), -(γ-Glu)-CO—(CH₂)_{q}—CO₂H];
   X₃₇ is P or K[(2-[2-(2-amino-ethoxy)-ethoxy]-acetyl), -(γ-Glu)-CO—(CH₂)_{q}—CO₂H];
   X₃₈ is P or K[(2-[2-(2-amino-ethoxy)-ethoxy]-acetyl), -(γ-Glu)-CO—(CH₂)_{q}—CO₂H];
   X₃₉ is selected from the group consisting of C, S, and K[(2-[2-(2-amino-ethoxy)-ethoxy]-acetyl)₂-(γ-Glu)-CO—(CH₂)_{q}—CO₂H];
   X₄₀ is selected from the group consisting of C and K[(2-[2-(2-amino-ethoxy)-ethoxy]-acetyl)₂-(γ-Glu)-CO—(CH₂)_{q}—CO₂H];
q is selected from the group consisting of 14, 15, 16, 17, 18, 19, and 20; and R₂ is a modification of the C-terminal group, wherein the modification is NH₂ or absent;
or a pharmaceutically acceptable salt thereof;
wherein if X₃₀ is G-R₂, then X₃₁ is absent;
wherein no more than one of X₁₀, X₁₂, X₁₃, X₁₄, X₁₆, X₁₇, X₁₉, X₂₀, X₂₁, X₂₃, X₂₄, X₂₆, X₂₇, X₂₈, X₂₉, X₃₀, X₃₁, X₃₂, X₃₃, X₃₄, X₃₅, X₃₆, X₃₇, X₃₈, X₃₉, and X₄₀ may be a substituent that contains a fatty acid; and
wherein no more than one of X₃₀, X₃₄, X₃₉, and X₄₀ may be C; and
wherein if one of X₃₀, X₃₄, X₃₉, and X₄₀ is C, then none of X₁₀, X₁₂, X₁₃, X₁₄, X₁₆, X₁₇, X₁₉, X₂₀, X₂₁, X₂₃, X₂₄, X₂₆, X₂₇, X₂₈, X₂₉, X₃₀, X₃₁, X₃₂, X₃₃, X₃₄, X₃₅, X₃₆, X₃₇, X₃₈, X₃₉, and X₄₀ is a substituent that contains a fatty acid;
wherein the GIP: GLP-1 Peptide has a receptor agonist potency ratio that is about 2.5:1 to about 10:1.

A further embodiment provides a novel GIP: GLP-1 Peptide of SEQ ID NO:3, wherein the GIP: GLP-1 Peptide has a GIP: GLP-1 receptor agonist potency ratio that is about 2.5:1 to about 5:1. A further embodiment of the present invention is a novel GIP: GLP-1 Peptide of SEQ ID NO:3, wherein the GIP: GLP-1 Peptide has a GIP: GLP-1 receptor agonist potency ratio that is about 2.5:1 to about 3.5:1.

The invention is further illustrated by the following compounds demonstrating the desired GIP potency ratio that is about 2.5:1 to about 10 times greater than the receptor potency at the GLP-1 receptor using the cAMP casein assay, however, these example peptides are not to be construed as limiting.

### PEPTIDE SYNTHESIS-Peptide 1

### Y-Aib-EGT-αMeF(2F)-TSDYSI-αMeL-LDEK ((2-[2-(2-Amino-ethoxy)-ethoxy]-acetyl)₂-(γGlu)-CO—(CH₂)₁₈—CO₂H)AQ-Aib-EFI-(D-Glu)-YLIEGGPSSGAPPPS-NH₂ (SEQ ID NO:4)

The structure of SEQ ID NO:4 is depicted below using the standard single letter amino acid codes with the exception of residues Aib2, αMeF(2F)6, αMeL13, K17, Aib20, D-Glu24, and Ser39 where the structures of these amino acid residues have been expanded:

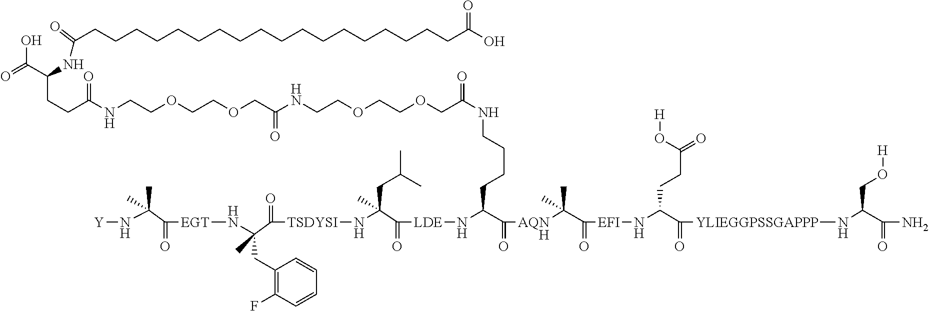

The peptide backbone of Peptide 1 is synthesized using Fluorenylmethyloxycarbonyl (Fmoc)/tert-Butyl (t-Bu) chemistry on a Symphony X peptide synthesizer (Gyros Protein Technologies. Tucson, AZ).

The resin consists of 1% DVB cross-linked polystyrene (Fmoc-Rink-MBHA Low Loading resin, 100-200 mesh, EMD Millipore) at a substitution of 0.3-0.4 meq/g. Standard side-chain protecting groups were used. Fmoc-Lys (Mtt)-OH is used for the lysine at position 17 and Boc-Tyr (tBu)-OH) was used for the tyrosine at position 1. Fmoc groups are removed prior to each coupling step (2×7 minutes) using 20% piperidine in DMF. All standard amino acid couplings are performed for 1 hour to a primary amine and 3 hour to a secondary amine, using an equal molar ratio of Fmoc amino acid (0.3 mM), diisopropylcarbodiimide (0.9 mM) and Oxyma (0.9 mM), at a 9-fold molar excess over the theoretical peptide loading. Exceptions are couplings to Co-methylated amino acids, which are coupled for 3 hours. After completion of the synthesis of the peptide backbone, the resin is thoroughly washed with DCM for 6 times to remove residual DMF. The Mtt protecting group on the lysine at position 17 is selectively removed from the peptide resin using two treatments of 30% hexafluoroisopropanol (Oakwood Chemicals) in DCM (2×40-minute treatment).

Subsequent attachment of the fatty acid-linker moiety is accomplished by coupling of 2-[2-(2-Fmoc-amino-ethoxy)-ethoxy]-acetic acid (Fmoc-AEEA-OH, ChemPep, Inc.), Fmoc-glutamic acid «-t-butyl ester (Fmoc-Glu-OtBu, Ark Pharm, Inc.), eicosanedioic acid (WuXi AppTec, Shanghai, China). 3-Fold excess of reagents (AA: PyAOP: DIPEA=1:1:1 mol/mol) are used for each coupling that is 1-hour long.

After the synthesis is complete, the peptide resin is washed with DCM, and then thoroughly air-dried. The dry resin is treated with 10 mL of cleavage cocktail (trifluoroacetic acid: water: triisopropylsilane, 95:2.5:2.5 v/v) for 2 hours at room temperature. The resin is filtered off, washed twice each with 2 mL of neat TFA, and the combined filtrates are treated with 5-fold excess volume of cold diethyl ether (−20° C.) to precipitate the crude peptide. The peptide/ether suspension is then centrifuged at 3500 rpm for 2 min to form a solid pellet, the supernatant is decanted, and the solid pellet is triturated with ether two additional times and dried in vacuo. The crude peptide is solubilized in 20% acetonitrile/20% Acetic acid/60% water and purified by RP-HPLC on a Luna 5 μm Phenyl-Hexyl preparative column (21×250 mm, Phenomenex) with linear gradients of 100% acetonitrile and 0.1% TFA/water buffer system (30-50% acetonitrile in 60 min). The purity of peptide is assessed using analytical RP-HPLC and pooling criteria is >95%. The main pool purity of compound 1 is found to be 98.0%. Subsequent lyophilization of the final main product pool yielded the lyophilized peptide TFA salt. The molecular weight is determined by LC-MS (obsd: M+3=1657.2; Calc M+3=1657.0).

### Peptides 2 Through Peptide 265

The compounds according to Peptide 2 (SEQ ID NO:5) through Peptide 265 (SEQ ID NO:139), shown in Table 1 below, are prepared substantially as described by the procedures of Peptide 1.

### Binding Assays

Glucagon (referred to as Gcg) is a Reference Standard prepared at Eli Lilly and Company. GLP-1, 7-36—NH₂ (referred to as GLP-1) is obtained from CPC Scientific (Sunnyvale, CA, 97.2% purity, 100 μM aliquots in 100% DMSO). GIP 1-42 (referred to as GIP) is prepared at Lilly Research Laboratories using peptide synthesis and HPLC chromatography as described above (>80% purity, 100 μM aliquots in 100% DMSO). [¹²⁵I]-radiolabeled Geg, GLP-1, or GIP is prepared using [¹²⁵I]-lactoperoxidase and obtained from Perkin Elmer (Boston, MA).

Stably transfected cell lines are prepared by subcloning receptor cDNA into a pcDNA3 expression plasmid and transfected into human embryonic kidney (HEK) 293 (hGcgR and hGLP-1R) or Chinese Hamster Ovary (CHO) (hGIPR) cells followed by selection with Geneticin (hGLP-1R and hGIPR) or hygromycin B (hGcgR).

Two methods are used for the preparation of crude cell membranes.

Method 1: Frozen cell pellets are lysed on ice in hypotonic buffer containing 50 mM Tris HCl, pH 7.5, and Roche Complete™ Protease Inhibitors with EDTA. The cell suspension is disrupted using a glass Potter-Elvehjem homogenizer fitted with a Teflon® pestle for 25 strokes. The homogenate is centrifuged at 4° C. at 1100×g for 10 minutes. The supernatant is collected and stored on ice while the pellets are resuspended in homogenization buffer and rehomogenized as described above. The homogenate is centrifuged at 1100×g for 10 minutes. The second supernatant is combined with the first supernatant and centrifuged at 35000×g for 1 hour at 4° C. The resulting membrane pellet is resuspended in homogenization buffer containing protease inhibitors at approximately 1 to 3 mg/mL, quick frozen in liquid nitrogen and stored as aliquots in a−80° C. freezer until use.

Method 2: Frozen cell pellets are lysed on ice in hypotonic buffer containing 50 mM Tris HCl, pH 7.5, 1 mM MgCl₂, Roche Complete™ EDTA-free Protease Inhibitors and 25 units/ml DNAse I (Invitrogen). The cell suspension is disrupted using a glass Potter-Elvehjem homogenizer fitted with a Teflon® pestle for 20 to 25 strokes. The homogenate is centrifuged at 4° C. at 1800×g for 15 minutes. The supernatant is collected and stored on ice while the pellets are resuspended in homogenization buffer (without DNAse I) and rehomogenized as described above. The homogenate is centrifuged at 1800×g for 15 minutes. The second supernatant is combined with the first supernatant and centrifuged an additional time at 1800×g for 15 minutes. The overall supernatant is then centrifuged at 25000×g for 30 minutes at 4° C. The resulting membrane pellet is resuspended in homogenization buffer (without DNAse I) containing protease inhibitors at approximately 1 to 3 mg/mL and stored as aliquots in a −80° C. freezer until use.

### Binding Determination Methods

The equilibrium binding dissociation constants (K_{d}) for the various receptor/radioligand interactions are determined from homologous competition binding analysis instead of saturation binding due to high propanol content in the [¹²⁵I] stock material. The K_{d} values determined for the receptor preparations were as follows: hGcgR (3.9 nM), hGLP-1R (1.2 nM) and hGIPR (0.14 nM).

### [¹²⁵I]-Glucagon Binding

The human Geg receptor binding assays are performed using a Scintillation Proximity Assay (SPA) format with wheat germ agglutinin (WGA) beads (Perkin Elmer). The binding buffer contains 25 mM 4-(2-hydroxyethyl)-1-piperazineethanesulfonic acid (HEPES), pH 7.4, 2.5 mM CaCl₂), 1 mM MgCl₂, 0.1% (w/v) bacitracin (Research Products), 0.003% (w/v) Polyoxyethylenesorbitan monolaurate (TWEEN®-20), and Roche Complete™ Protease Inhibitors without EDTA. Peptides and Gog are thawed and 3-fold serially diluted in 100% DMSO (10 point concentration response curves). Next, 5 μL serially diluted compound or DMSO is transferred into Corning® 3632 clear bottom assay plates containing 45 μL assay binding buffer or unlabeled Geg control (non-specific binding or NSB, at 1 μM final). Then, 50 μL [¹²⁵I]-Gcg (0.15 nM final), 50 μL human GcgR membranes (1.5 μg/well) and 50 μL of WGA SPA beads (80 to 150 μg/well) are added with a Biotek Multiflo dispenser. Plates are sealed and mixed on a plate shaker (setting 6) for 1 minute and read with a PerkinElmer Trilux MicroBeta® scintillation counter after 12 hours of incubation/settling time at room temperature. Final assay concentration ranges for peptides tested in response curves is typically 1150 nM to 0.058 nM and for the control Geg from 1000 nM to 0.05 nM.

### [¹²⁵I]-GLP-1 Binding

The human GLP-1 receptor binding assay is performed using an SPA format with WGA beads. The binding buffer contains 25 mM HEPES, pH 7.4, 2.5 mM CaCl₂, 1 mM MgCl₂, 0.1% (w/v) bacitracin, 0.003% (w/v) TWEEN®-20, and Roche Complete™ Protease Inhibitors without EDTA. Peptides and GLP-1 are thawed and 3-fold serially diluted in 100% DMSO (10 point concentration response curves). Next, 5 μL serially diluted compound or DMSO is transferred into Corning® 3632 clear bottom assay plates containing 45 μL assay binding buffer or unlabeled GLP-1 control (non-specific binding or NSB, at 0.25 μM final). Then, 50 μL [¹²⁵I]-GLP-1 (0.15 nM final), 50 μL human GLP-1R membranes (0.5 μg/well and 50 μL of WGA SPA beads (100 to 150 μg/well) are added with a Biotek Multiflo dispenser. Plates are sealed and mixed on a plate shaker (setting 6) for 1 minute and read with a PerkinElmer Trilux MicroBeta® scintillation counter after 5 to 12 hours of incubation/settling time at room temperature. Final assay concentration ranges for peptides tested in response curves are typically 1150 nM to 0.058 nM and for the control GLP-1, 250 nM to 0.013 nM.

### [¹²⁵I]-GIP Binding

The human GIP receptor binding assay is performed using an SPA format with WGA beads. The binding buffer contains 25 mM HEPES, pH 7.4, 2.5 mM CaCl₂, 1 mM MgCl₂, 0.1% (w/v) bacitracin, 0.003% (w/v) TWEEN®-20, and Roche Complete™ Protease Inhibitors without EDTA. Peptides and GIP are thawed and 3 fold serially diluted in 100% DMSO (10 point concentration response curves). Next, 5 μL serially diluted compound or DMSO is transferred into Corning® 3632 clear bottom assay plates containing 45 μL assay binding buffer or unlabeled GIP control (non-specific binding or NSB, at 0.25 μM final). Then, 50 L [¹²⁵I]-GIP (0.075-0.15 nM final), 50 μL human GIPR membranes (3 μg/well) and 50 μL of WGA SPA beads (100 to 150 μg/well) are added with a Biotek Multiflo dispenser. Plates are sealed and mixed on a plate shaker (setting 6) for 1 minute and read with a PerkinElmer Trilux MicroBeta® scintillation counter after 2.5 to 12 hours of incubation/settling time at room temperature. Final assay concentration ranges for peptides tested in response curves is typically 1150 to 0.058 nM or 115 nM to 0.0058 nM and for the control GIP, 250 nM to 0.013 nM.

### Binding Assay Data Normalization

Raw CPM data for concentration curves of peptides, Geg, GLP-1, or GIP are converted to percent inhibition by subtracting nonspecific binding (binding in the presence of excess unlabeled Geg, GLP-1, or GIP, respectively) from the individual CPM values and dividing by the total binding signal, also corrected by subtracting nonspecific binding. Data are analyzed using four-parameter (curve maximum, curve minimum, IC₅₀, Hill slope) nonlinear regression routines (Genedata Screener, version 12.0.4, Genedata AG, Basal, Switzerland). The affinity constant (Kᵢ) is calculated from the absolute IC₅₀ value based upon the equation Kᵢ=IC₅₀/(1+D/K_{d}) where D=the concentration of radioligand used in the experiment, IC₅₀ is the concentration causing 50% inhibition of binding and Kais the equilibrium binding dissociation constant of the radioligand (described above). Values for Kᵢ are reported as the geometric mean, with error expressed as the standard error of the mean (SEM) and n is equal to the number of independent replicates (determined in assays performed on different days). Geometric Means are calculated as follows:

Geometric Mean=10 (Arithmetic Mean of Log Ki Values))

The Ki Ratio (Ki for native control peptide/Ki for test compound) at each receptor and each species is calculated. The Ki Ratio is a rapid indication of the apparent affinity of a peptide compared to the native control peptide. A Ki Ratio <1 indicates that the test peptide has a lower affinity (higher Ki value) for the receptor than the native peptide, whereas a Ki Ratio >1 indicates that the test peptide has a higher affinity (lower Ki value) for the receptor than the native peptide.

### EXAMPLE 1

### CAMP Pharmacological Functional Assay in Presence of Casein

An additional set of cAMP assays are conducted in HEK293 cells expressing the human GLP-1 receptor (GLP-1R), gastric inhibitory peptide receptor (GIPR), Glucagon receptor (GcgR). Pharmacological activity of the hGLP1R/GIPR peptides are determined in HEK293 cells stably expressing the human GLP-1 receptor (GLP-1R), gastric inhibitory peptide receptor (GIPR), or GLP-2 receptor (GLP-2R). Each receptor over-expressing cell line (20 μl) is treated with the test peptide in DMEM (Gibco Cat #31053) supplemented with 0.1% Casein (Sigma Cat #C4765), 250 μM IBMX, 1X GlutaMAX™ (Gibco Cat #35050), and 20 mM HEPES (HyClone Cat #SH30237.01) in a 20 μl assay volume. After 60 minute incubation at room temperature, the resulting increase in intracellular cAMP is quantitatively determined using the CisBio CAMP Dynamic 2 HTRF Assay Kit (62AM4PEJ). The Lysis buffer containing cAMP-d2 conjugate (20 μl) and the antibody anti-cAMP-Eu3+-Cryptate (20 μl) are then added to determine the CAMP level. After 1 hour-incubation at room temperature, HTRF signal is detected with an Envision 2104 plate reader (PerkinElmer). Each of the two incubation steps (60 minutes and 1 hour) may be conducted at about room temperature or about 37C, so long as both the 60 minute and 1 hour incubations are completed at about the same temperature for each run of the assay. Fluorescent emission at 620 nm and at 665 nm is measured and the ratio between 620 nm and at 665 nm is calculated and then are converted to nM CAMP per well using a cAMP standard curve. Dose response curves of compounds are plotted as the percentage of stimulation normalized to minimum (buffer only) and maximum (maximum concentration of each control ligand) values and analyzed using a four parameter non-liner regression fit with a variable slope (Genedata Screener 13). EC50 is the concentration of compound causing half-maximal simulation in a dose response curve. A relative EC₅₀ value is derived by non-linear regression analysis using the percent maximal response vs. the concentration of peptide added, fitted to a four-parameter logistic equation.

Using Homogeneous Time Resolved Fluorescence methods, assays are conducted to determine the intrinsic potency of Example and comparator molecules performed in the presence of casein (instead of serum albumin) as a nonspecific blocker, which does not interact with the fatty acid moieties of the analyzed molecules.

Intracellular cAMP levels are determined by extrapolation using a standard curve. Dose response curves of compounds are plotted as the percentage of stimulation normalized to minimum (buffer only) and maximum (maximum concentration of each control ligand) values and analyzed using a four parameter non-linear regression fit with a variable slope (Genedata Screener 13). EC50 is the concentration of compound causing half-maximal simulation in a dose response curve. Each relative EC50 value for the Geometric mean calculation is determined from a curve fitting.

Concentration response curves of compounds are plotted as the percentage of stimulation normalized to minimum (buffer only) and maximum (maximum concentration of each control ligand) values and analyzed using a four parameter non-liner regression fit with a variable slope (Genedata Screener 13). EC50 is the concentration of compound causing half-maximal simulation in a dose response curve.

The EC₅₀ summary statistics are computed as follows:

Geometric mean:

GM=10∧(arithmetic mean of log₁₀ transformed EC₅₀ values).

The standard error of the mean is reported:

SEM=geometric mean×(standard deviation of log₁₀ transformed EC₅₀ values/square root of the #of runs)×logₑ of 10.

The log transform accounts for the EC₅₀ values falling on a multiplicative, rather than an arithmetic scale.

Each day the assay is run, the test peptides are run plus the native ligands GIP and GLP-1 and buffer only as baseline (minimum), and the highest concentration of the respective GIP and GLP-1 standard that day is used as a maximum for the calculation.

TABLE 1
Functional activation of hGLP-1R and hGIPR in the presence of 0.1% Casein.

| | | | hGIPR | hGLP1R | [hGIPR/ |
|---|---|---|---|---|---|
| | | SEQ | CAMP | CAMP | hGLP1R] |
| Peptide | | ID | EC₅₀ | EC₅₀ | EC₅₀ |
| Number | Compound Name | NO | ratio | ratio | Ratio |
| 1 | Y-Aib-EGT-αMeF(2F)-TSDYSI-αMeL- | 4 | 4.65 | 1.12 | 4.15 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)-YLIEGGPSSGAPPPS- | | | | |
| | NH₂ | | | | |
| 2 | Y-Aib-EGT-αMeF(2F)-TSDYSI-αMeL- | 5 | 5.89 | 0.888 | 6.63 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-EFI-(D- | | | | |
| | Glu)-YLIEGGPSSGAPPPS-NH₂ | | | | |
| 3 | Y-Aib-EGT-αMeF(2F)-TSDYSI-αMeL- | 6 | 4.51 | 1.25 | 3.61 |
| | LD-Orn-K((2-[2-(2-Amino-ethoxy)- | | | | |
| | ethoxy]-acetyl)₂-(γGlu)- | | | | |
| | CO—(CH₂)₁₈—CO₂H)AQ-Aib-EFI-(D- | | | | |
| | Glu)-YLIEGGPSSGAPPPS-NH₂ | | | | |
| 4 | Y-Aib-EGT-αMeF(2F)-TSD-4Pal-SI- | 7 | 5.95 | 1.41 | 4.22 |
| | αMeL-LD-Orn-K((2-[2-(2-Amino- | | | | |
| | ethoxy)-ethoxy]-acetyl)₂-(γGlu)- | | | | |
| | CO—(CH₂)₁₆—CO₂H)AQ-Aib-EFI-(D- | | | | |
| | Glu)-αMeY-LIEGGPSSGAPPPS-NH₂ | | | | |
| 8 | Y-Aib-EGTFTSDYSILLDKK((2-[2-(2- | 8 | 1.97 | 0.419 | 4.70 |
| | Amino-ethoxy)-ethoxy]-acetyl)₂-(γGlu)- | | | | |
| | CO—(CH₂)₁₈—CO₂H)AQ-Aib- | | | | |
| | AFIEYLIEGGPSSGAPPPS-NH₂ | | | | |
| 9 | Y-Aib-EGTFTSDYSILLDSK((2-[2-(2- | 9 | 0.768 | 0.314 | 2.45 |
| | Amino-ethoxy)-ethoxy]-acetyl)₂-(γGlu)- | | | | |
| | CO—(CH₂)₁₈—CO₂H)AQ-Aib- | | | | |
| | AFIEYLIEGGPSSGAPPPS-NH₂ | | | | |
| 20 | Y-Aib-EGTFTSDYSI-αMeL-LDKK((2- | 10 | 2.81 | 0.577 | 4.87 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGGPSSGAPPPS-NH₂ | | | | |
| 21 | Y-Aib-EGTFTSDYSI-αMeL-LDEK((2- | 11 | 1.95 | 0.402 | 4.85 |
| | [2-(2-Amino-ethoxy)-ethoxy]-acetyl)₂- | | | | |
| | (γGlu)-CO—(CH₂)₁₈—CO₂H)AQ-αMeK- | | | | |
| | AFIEYLLEGGPSSGAPPPS-NH₂ | | | | |
| 22 | Y-Aib-EGTFTSDYSI-αMeL-LDEK((2- | 12 | 1.86 | 0.29 | 6.41 |
| | [2-(2-Amino-ethoxy)-ethoxy]-acetyl)₂- | | | | |
| | (γGlu)-CO—(CH₂)₁₈—CO₂H)AQ-αMeK- | | | | |
| | AFIEYLLE-Aib-GPSSGAPPPS-NH₂ | | | | |
| 25 | Y-Aib-EGTFTSDYSK((2-[2-(2-Amino- | 13 | 0.636 | 0.197 | 3.23 |
| | ethoxy)-ethoxy]-acetyl)₂-(γGlu)- | | | | |
| | CO—(CH₂)₁₈—CO₂H)LLDKIAQ-Aib- | | | | |
| | AFIEYLIEGGPSSGAPPPS-NH₂ | | | | |
| 26 | Y-Aib-EGTFTSDYSIK((2-[2-(2-Amino- | 14 | 0.585 | 0.238 | 2.46 |
| | ethoxy)-ethoxy]-acetyl)₂-(γGlu)- | | | | |
| | CO—(CH₂)₁₈—CO₂H)LDKIAQ-Aib- | | | | |
| | AFIEYLIEGGPSSGAPPPS-NH₂ | | | | |
| 29 | Y-Aib-EGTFTSDYSILLDK((2-[2-(2- | 15 | 0.536 | 0.0671 | 7.99 |
| | Amino-ethoxy)-ethoxy]-acetyl)₂-(γGlu)- | | | | |
| | CO—(CH₂)₁₈—CO₂H)IAQ-Aib- | | | | |
| | AFIEYLIEGGPSSGAPPPS-NH₂ | | | | |
| 31 | Y-Aib-EGTFTSDYSILLDKIAK((2-[2- | 16 | 0.456 | 0.0708 | 6.44 |
| | (2-Amino-ethoxy)-ethoxy]-acetyl)₂- | | | | |
| | (γGlu)-CO—(CH₂)₁₈—CO₂H)-Aib- | | | | |
| | AFIEYLIEGGPSSGAPPPS-NH₂ | | | | |
| 32 | Y-Aib-EGTFTSDYSILLDKIAQ-Aib- | 17 | 0.84 | 0.136 | 6.18 |
| | K((2-[2-(2-Amino-ethoxy)-ethoxy]- | | | | |
| | acetyl)₂-(γGlu)-CO—(CH₂)₁₈—CO₂H) | | | | |
| | FIEYLIEGGPSSGAPPPS-NH₂ | | | | |
| 33 | Y-Aib-EGTFTSDYSILLDKIAQ-Aib- | 18 | 0.00222 | 0.000256 | 8.67 |
| | AFK((2-[2-(2-Amino-ethoxy)-ethoxy]- | | | | |
| | acetyl)₂-(γGlu)-CO—(CH₂)₁₈—CO₂H) | | | | |
| | EYLIEGGPSSGAPPPS-NH₂ | | | | |
| 34 | Y-Aib-EGTFTSDYSILLDKIAQ-Aib- | 19 | 0.393 | 0.0392 | 10.03 |
| | AFIK((2-[2-(2-Amino-ethoxy)-ethoxy]- | | | | |
| | acetyl)₂-(γGlu)-CO—(CH₂)₁₈—CO₂H) | | | | |
| | YLIEGGPSSGAPPPS-NH₂ | | | | |
| 37 | Y-Aib-EGTFTSDYSILLDKIAQ-Aib- | 20 | 0.532 | 0.0533 | 9.98 |
| | AFIEYLIK((2-[2-(2-Amino-ethoxy)- | | | | |
| | ethoxy]-acetyl)₂-(γGlu)- | | | | |
| | CO—(CH₂)₁₈—CO₂H)GGPSSGAPPPS- | | | | |
| | NH₂ | | | | |
| 41 | Y-Aib-EGTFTSDYSILLDKIAQ-Aib- | 21 | 0.637 | 0.0637 | 10.00 |
| | AFIEYLIEGGPK((2-[2-(2-Amino- | | | | |
| | ethoxy)-ethoxy]-acetyl)₂-(γGlu)- | | | | |
| | CO—(CH₂)₁₈—CO₂H)SGAPPPS-NH₂ | | | | |
| 46 | Y-Aib-EGTFTSDYSILLDKIAQ-Aib- | 22 | 0.828 | 0.0969 | 8.54 |
| | AFIEYLIEGGPSSGAPK((2-[2-(2- | | | | |
| | Amino-ethoxy)-ethoxy]-acetyl)₂-(γGlu)- | | | | |
| | CO—(CH₂)₁₈—CO₂H)PS-NH₂ | | | | |
| 47 | Y-Aib-EGTFTSDYSILLDKIAQ-Aib- | 23 | 0.654 | 0.089 | 7.35 |
| | AFIEYLIEGGPSSGAPPK((2-[2-(2- | | | | |
| | Amino-ethoxy)-ethoxy]-acetyl)₂-(γGlu)- | | | | |
| | CO—(CH₂)₁₈—CO₂H)S-NH₂ | | | | |
| 48 | Y-Aib-EGTFTSDYSILLDKIAQ-Aib- | 23 | 0.863 | 0.0966 | 8.93 |
| | AFIEYLIEGGPSSGAPPPK((2-[2-(2- | | | | |
| | Amino-ethoxy)-ethoxy]-acetyl)₂-(γGlu)- | | | | |
| | CO—(CH₂)₁₈—CO₂H)—NH₂ | | | | |
| 50 | Y-Aib-EGTFTSDYSILLDKK((2-[2-(2- | 25 | 1.96 | 0.675 | 2.90 |
| | Amino-ethoxy)-ethoxy]-acetyl)₂-(γGlu)- | | | | |
| | CO—(CH₂)₁₈—CO₂H)AQ-Aib- | | | | |
| | AFIEYLIEGGPSSG-NH₂ | | | | |
| 51 | Y-Aib-EGTFTSDYSILLDKK((2-[2-(2- | 26 | 1.69 | 0.426 | 3.97 |
| | Amino-ethoxy)-ethoxy]-acetyl)₂-(γGlu)- | | | | |
| | CO—(CH₂)₁₈—CO₂H)AQ-Aib- | | | | |
| | AFIEYLIEGG-NH₂ | | | | |
| 52 | Y-Aib-EGTFTSDYSI-αMeL-LDSK((2- | 27 | 1.4 | 0.514 | 2.72 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGGPSSGAPPPS-NH₂ | | | | |
| 60 | Y-Aib-EGTFTSDYSI-αMeL-LD-Aib- | 28 | 1.06 | 0.237 | 4.47 |
| | K((2-[2-(2-Amino-ethoxy)-ethoxy]- | | | | |
| | acetyl)₂-(γGlu)-CO—(CH₂)₁₈—CO₂H)AQ- | | | | |
| | Aib-AFIEYLIEGGPSSGAPPPS-NH₂ | | | | |
| 62 | Y-Aib-EGTFTSDYSI-αMeL-LDKK((2- | 29 | 2.33 | 0.463 | 5.03 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGG-NH₂ | | | | |
| 63 | Y-Aib-EGTFTSDYSI-αMeL-LDEK((2- | 30 | 1.58 | 0.386 | 4.09 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGG-NH₂ | | | | |
| 64 | Y-Aib-EGTFTSDYSI-αMeL-LDSK((2- | 31 | 1.57 | 0.429 | 3.66 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGG-NH₂ | | | | |
| 65 | Y-Aib-EGTFTSDYSI-αMeL-LDTK((2- | 32 | 1.04 | 0.233 | 4.46 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGG-NH₂ | | | | |
| 66 | Y-Aib-EGTFTSDYSI-αMeL-LDEK((2- | 33 | 1.89 | 0.255 | 7.41 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLLEGGPSSGAPPPS-NH₂ | | | | |
| 68 | Y-Aib-EGTFTSDY-αMeS-ILLDKK((2- | 34 | 1.67 | 0.354 | 4.72 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGGPSSGAPPPS-NH₂ | | | | |
| 69 | Y-Aib-EGT-αMeF-TSDYSI-αMeL- | 35 | 2.73 | 0.85 | 3.21 |
| | LDKK((2-[2-(2-Amino-ethoxy)-ethoxy]- | | | | |
| | acetyl)₂-(γGlu)-CO—(CH₂)₁₈—CO₂H)AQ- | | | | |
| | Aib-AFIEYLIEGGPSSGAPPPS-NH₂ | | | | |
| 73 | Y-Aib-EGTFTSDYSILLDKIAQ-Aib- | 36 | 0.174 | 0.0225 | 7.73 |
| | AFIEYLIK((2-[2-(2-Amino-ethoxy)- | | | | |
| | ethoxy]-acetyl)₂-(γGlu)- | | | | |
| | CO—(CH₂)₁₈—CO₂H)GG-NH₂ | | | | |
| 80 | Y-(D-Ala)-EGTFTSDYSILLDKK((2-[2- | 37 | 1.09 | 0.227 | 4.80 |
| | (2-Amino-ethoxy)-ethoxy]-acetyl)₂- | | | | |
| | (γGlu)-CO—(CH₂)₁₈—CO₂H)AQ-Aib- | | | | |
| | AFIEYLIEGGPSSGAPPPS-NH₂ | | | | |
| 81 | Y-Aib-EGTFTSDY-(D-Ser)- | 38 | 0.373 | 0.063 | 5.92 |
| | ILLDKK((2-[2-(2-Amino-ethoxy)- | | | | |
| | ethoxy]-acetyl)₂-(γGlu)- | | | | |
| | CO—(CH₂)₁₈—CO₂H)AQ-Aib- | | | | |
| | AFIEYLIEGGPSSGAPPPS-NH₂ | | | | |
| 83 | Y-Aib-EGTFTSDYSILLDKK((2-[2-(2- | 39 | 0.804 | 0.166 | 4.84 |
| | Amino-ethoxy)-ethoxy]-acetyl)₂-(γGlu)- | | | | |
| | CO—(CH₂)₁₈—CO₂H)AQ-Aib-AFIEYLI- | | | | |
| | (D-Glu)-GGPSSGAPPPS-NH₂ | | | | |
| 84 | Y-Aib-EGTFTSDYSILLDKK((2-[2-(2- | 40 | 1.59 | 0.173 | 9.19 |
| | Amino-ethoxy)-ethoxy]-acetyl)₂-(γGlu)- | | | | |
| | CO—(CH₂)₁₈—CO₂H)AQ-Aib-AFI-(D- | | | | |
| | Glu)-YLIEGGPSSGAPPPS-NH₂ | | | | |
| 88 | Y-Pro-EGTFTSDYSILLDKK((2-[2-(2- | 41 | 0.392 | 0.0918 | 4.27 |
| | Amino-ethoxy)-ethoxy]-acetyl)₂-(γGlu)- | | | | |
| | CO—(CH₂)₁₈—CO₂H)AQ-Aib- | | | | |
| | AFIEYLIEGGPSSGAPPPS-NH₂ | | | | |
| 89 | Y-Aib-Aad-GTFTSDYSILLDKK((2-[2- | 42 | 1.57 | 0.175 | 8.97 |
| | (2-Amino-ethoxy)-ethoxy]-acetyl)₂- | | | | |
| | (γGlu)-CO—(CH₂)₁₈—CO₂H)AQ-Aib- | | | | |
| | AFIEYLIEGGPSSGAPPPS-NH₂ | | | | |
| 95 | Y-Aib-EGT-αMeF-TSDYSILLDKIAQ- | 43 | 0.145 | 0.0576 | 2.52 |
| | Aib-AFIEYLIK((2-[2-(2-Amino- | | | | |
| | ethoxy)-ethoxy]-acetyl)₂-(γGlu)- | | | | |
| | CO—(CH₂)₁₈—CO₂H)GG-NH₂ | | | | |
| 97 | Y-Aib-EGTFTSDYSI-αMeL-LDKIAQ- | 44 | 0.0953 | 0.0268 | 3.56 |
| | Aib-AFIEYLIK((2-[2-(2-Amino- | | | | |
| | ethoxy)-ethoxy]-acetyl)₂-(γGlu)- | | | | |
| | CO—(CH₂)₁₈—CO₂H)GG-NH₂ | | | | |
| 98 | Y-Aib-EGT-αMeF-TSDYSILLDKK((2- | 45 | 2.43 | 0.384 | 6.33 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGGPSSG-NH₂ | | | | |
| 99 | Y-Aib-EGT-αMeF-TSDYSI-αMeL- | 46 | 2.27 | 0.629 | 3.61 |
| | LDSK((2-[2-(2-Amino-ethoxy)-ethoxy]- | | | | |
| | acetyl)₂-(γGlu)-CO—(CH₂)₁₈—CO₂H)AQ- | | | | |
| | Aib-AFIEYLIEGGPSSG-NH₂ | | | | |
| 103 | Y-Aib-EGT-αMeF(2F)-TSDYSI-αMeL- | 47 | 2.01 | 0.655 | 3.07 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFIEYLIEGG-NH₂ | | | | |
| 104 | Y-Aib-EGT-αMeF(2F)-TSDYSI-αMeL- | 48 | 4.93 | 1.85 | 2.66 |
| | LDEK((2-[2-(2-Amino-ethoxy)-ethoxy]- | | | | |
| | acetyl)₂-(γGlu)-CO—(CH₂)₁₆—CO₂H)AQ- | | | | |
| | Aib-EFIEYLIEGG-NH₂ | | | | |
| 108 | Y-Aib-EGTFTSDYSILLDKK((2-[2-(2- | 49 | 2.19 | 0.218 | 10.05 |
| | Amino-ethoxy)-ethoxy]-acetyl)-AOC- | | | | |
| | (γGlu)-CO—(CH₂)₁₈—CO₂H)AQ-Aib- | | | | |
| | AFIEYLIEGGPSSGAPPPS-NH₂ | | | | |
| 109 | Y-Aib-EGTFTSDYSILLDKK(AOC-(2- | 50 | 1.83 | 0.182 | 10.05 |
| | [2-(2-Amino-ethoxy)-ethoxy]-acetyl)- | | | | |
| | (γGlu)-CO—(CH₂)₁₈—CO₂H)AQ-Aib- | | | | |
| | AFIEYLIEGGPSSGAPPPS-NH2 | | | | |
| 110 | Y-Aib-EGTFTSDYSILLDKK((2-[2-(2- | 51 | 0.929 | 0.358 | 2.59 |
| | Amino-ethoxy)-ethoxy]-acetyl)-(γGlu)- | | | | |
| | (Trx)-CO—(CH₂)₁₈—CO₂H)AQ-Aib- | | | | |
| | AFIEYLIEGGPSSGAPPPS-NH₂ | | | | |
| 111 | Y-Aib-EGTFTSDYSILLDKK((2-[2-(2- | 52 | 1.1 | 0.209 | 5.26 |
| | Amino-ethoxy)-ethoxy]-acetyl)-(Trx)- | | | | |
| | (γGlu)-CO—(CH₂)₁₈—CO₂H)AQ-Aib- | | | | |
| | AFIEYLIEGGPSSGAPPPS-NH₂ | | | | |
| 112 | Y-Aib-EGTFTSDYSILLDKK((2-[2-(2- | 53 | 1.53 | 0.402 | 3.81 |
| | Amino-ethoxy)-ethoxy]-acetyl)-(εK)- | | | | |
| | (γGlu)-CO—(CH₂)₁₈—CO₂H)AQ-Aib- | | | | |
| | AFIEYLIEGGPSSGAPPPS-NH₂ | | | | |
| 113 | Y-Aib-EGTFTSDYSILLDKK((2-[2-(2- | 54 | 1.39 | 0.275 | 5.05 |
| | Amino-ethoxy)-ethoxy]-acetyl)₂-(εK)- | | | | |
| | CO—(CH₂)₁₈—CO₂H)AQ-Aib- | | | | |
| | AFIEYLIEGGPSSGAPPPS-NH₂ | | | | |
| 114 | Y-Aib-EGTFTSDYSILLDKK((2-[2-(2- | 55 | 1.65 | 0.234 | 7.05 |
| | Amino-ethoxy)-ethoxy]-acetyl)-(εK)- | | | | |
| | (εK)-CO—(CH₂)₁₈—CO₂H)AQ-Aib- | | | | |
| | AFIEYLIEGGPSSGAPPPS-NH₂ | | | | |
| 115 | Y-Aib-EGT-αMeF-TSDYSI-αMeL- | 56 | 1.85 | 0.743 | 2.49 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-AFIEYLIEGGPSSGAPPPS-NH₂ | | | | |
| 118 | Y-Aib-EGT-αMeF(2F)-TSDYSI-αMeL- | 57 | 3.42 | 1.13 | 3.03 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFIEYLIEGGPSSGAPPPS-NH₂ | | | | |
| 120 | Y-Aib-cTA-GT-αMeF-TSDYSI-αMeL- | 58 | 1.67 | 0.319 | 5.24 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-AFIEYLIEGGPSSGAPPPS-NH₂ | | | | |
| 123 | Y-Aib-EGT-αMeF-TSDYSI-αMeL- | 59 | 4.04 | 1.58 | 2.56 |
| | LDKK((2-[2-(2-Amino-ethoxy)-ethoxy]- | | | | |
| | acetyl)₂-(γGlu)-CO—(CH₂)₁₈—CO₂H)AQ- | | | | |
| | Aib-EFIEYLIEGGPSSG-NH₂ | | | | |
| 125 | Y-Aib-EGT-αMeF-TSDYSI-αMeL- | 60 | 3.79 | 1.31 | 2.89 |
| | LDKK((2-[2-(2-Amino-ethoxy)-ethoxy]- | | | | |
| | acetyl)₂-(γGlu)₂-CO—(CH₂)₁₈—CO₂H)AQ- | | | | |
| | Aib-AFIEYLIEGGPSSGAPPPS-NH₂ | | | | |
| 126 | Y-Aib-EGT-αMeF-TSDYSI-αMeL- | 61 | 2.53 | 0.869 | 2.91 |
| | LDKK((2-[2-(2-Amino-ethoxy)-ethoxy]- | | | | |
| | acetyl)₂-(γGlu)-CO—(CH₂)₁₈—CO₂H)AQ- | | | | |
| | Aib-AFIEYLIEAGPSSGAPPPS-NH₂ | | | | |
| 128 | Y-Aib-EGT-αMeF-TSDHSILLDKK((2- | 62 | 2.46 | 0.7 | 3.51 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGGPSSGAPPPS-NH₂ | | | | |
| 129 | Y-Aib-EGT-αMeF-TSDLSILLDKK((2- | 63 | 1.88 | 0.543 | 3.46 |
| | [2-(2-Amino-ethoxy)-ethoxy]-acetyl)₂- | | | | |
| | (γGlu)-CO—(CH₂)₁₈—CO₂H)AQ-Aib- | | | | |
| | AFIEYLIEGGPSSGAPPPS-NH₂ | | | | |
| 137 | Y-Aib-EGT-αMeF-TSDYSI-αMeL- | 64 | 4.47 | 1.25 | 3.58 |
| | LDKK((2-[2-(2-Amino-ethoxy)-ethoxy]- | | | | |
| | acetyl)₂-(γGlu)-CO—(CH₂)₁₈—CO₂H)AQ- | | | | |
| | Aib-Aad-FIEYLIEGGPSSGAPPPS-NH₂ | | | | |
| 139 | Y-Aib-EGT-αMeF-TSDYSI-αMeL- | 65 | 3.61 | 1.13 | 3.19 |
| | LDKK((2-[2-(2-Amino-ethoxy)-ethoxy]- | | | | |
| | acetyl)₂-(γGlu)-CO—(CH₂)₁₈—CO₂H)AQ- | | | | |
| | Aib-TFIEYLIEGGPSSGAPPPS-NH₂ | | | | |
| 140 | Y-Aib-EGT-αMeF-TSDYSI-αMeL- | 66 | 3.76 | 1.16 | 3.24 |
| | LDKK((2-[2-(2-Amino-ethoxy)-ethoxy]- | | | | |
| | acetyl)₂-(γGlu)-CO—(CH₂)₁₈—CO₂H)AQ- | | | | |
| | Aib-DFIEYLIEGGPSSGAPPPS-NH₂ | | | | |
| 143 | Y-Aib-EGT-αMeF-TSDYSI-αMeL- | 67 | 2.78 | 0.714 | 3.89 |
| | LDKK((2-[2-(2-Amino-ethoxy)-ethoxy]- | | | | |
| | acetyl)₂-(γGlu)-CO—(CH₂)₁₈—CO₂H)AQ- | | | | |
| | Aib-Aib-FIEYLIEGGPSSGAPPPS-NH₂ | | | | |
| 144 | Y-Aib-EGT-αMeF-TSDYSI-αMeL- | 68 | 3.6 | 0.851 | 4.23 |
| | LDKK((2-[2-(2-Amino-ethoxy)-ethoxy]- | | | | |
| | acetyl)₂-(γGlu)- | | | | |
| | CO—(CH₂)₁₈—CO₂H)AQH-Aib- | | | | |
| | FIEYLIEGGPSSGAPPPS-NH₂ | | | | |
| 147 | Y-Aib-EGT-αMeF(2F)-TSDYSI-αMeL- | 69 | 4.14 | 1.13 | 3.66 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-Aad-YLIEGGPSSGAPPPS- | | | | |
| | NH₂ | | | | |
| 148 | Y-Aib-EGT-αMeF(2F)-TSDYSI-αMeL- | 70 | 2.7 | 0.859 | 3.14 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFIAYLIEGGPSSGAPPPS-NH₂ | | | | |
| 149 | Y-Aib-EGT-αMeF(2F)-TSDYSI-αMeL- | 71 | 1.82 | 0.484 | 3.76 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFIVYLIEGGPSSGAPPPS-NH₂ | | | | |
| 150 | Y-Aib-EGT-αMeF(2F)-TSDYSI-αMeL- | 72 | 2.64 | 0.79 | 3.34 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFISYLIEGGPSSGAPPPS-NH₂ | | | | |
| 151 | Y-Aib-EGT-αMeF(2F)-TSDYSI-αMeL- | 73 | 0.262 | 0.0278 | 9.42 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFIPYLIEGGPSSGAPPPS-NH₂ | | | | |
| 152 | Y-Aib-EGT-αMeF(2F)-TSDYSI-αMeL- | 74 | 2.57 | 0.484 | 5.31 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-Aib-YLIEGGPSSGAPPPS-NH₂ | | | | |
| 153 | Y-Aib-EGT-αMeF(2F)-TSDYSI-αMeL- | 75 | 1.7 | 0.501 | 3.39 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFIHYLIEGGPSSGAPPPS-NH₂ | | | | |
| 154 | Y-Aib-EGT-αMeF(2F)-TSDHSI-αMeL- | 76 | 5.9 | 1.23 | 4.80 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFIEYLIEGGPSSGAPPPS-NH₂ | | | | |
| 155 | Y-Aib-EGT-αMeF(2F)-TSD-cTA-SI- | 77 | 0.584 | 0.0978 | 5.97 |
| | αMeL-LDEK((2-[2-(2-Amino-ethoxy)- | | | | |
| | ethoxy]-acetyl)₂-(γGlu)- | | | | |
| | CO—(CH₂)₁₈—CO₂H)AQ-Aib- | | | | |
| | EFIEYLIEGGPSSGAPPPS-NH₂ | | | | |
| 157 | Y-Aib-EGT-αMeF(2F)-TSD-3Pal-SI- | 78 | 3.15 | 1.25 | 2.52 |
| | αMeL-LDEK((2-[2-(2-Amino-ethoxy)- | | | | |
| | ethoxy]-acetyl)₂-(γGlu)- | | | | |
| | CO—(CH₂)₁₈—CO₂H)AQ-Aib- | | | | |
| | EFIEYLIEGGPSSGAPPPS-NH₂ | | | | |
| 167 | Y-Aib-EGT-αMeF(2F)-TSDHSI-αMeL- | 79 | 0.291 | 0.0487 | 5.98 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-TFI-(D-Glu)-YLIEGGPSSG-NH₂ | | | | |
| 168 | Y-Aib-EGT-αMeF(2F)-TSDHSI-αMeL- | 80 | 0.313 | 0.0323 | 9.69 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-TFI-(D-Glu)-YLIEGGPSSGAPPPS- | | | | |
| | NH₂ | | | | |
| 169 | Y-Aib-EGT-αMeF(2F)-TSDHSI-αMeL- | 81 | 0.122 | 0.0136 | 8.97 |
| | LDEK((2-[2-(2-Amino-ethoxy)-ethoxy]- | | | | |
| | acetyl)₂-(γGlu)-CO—(CH₂)₁₆—CO₂H)AQ- | | | | |
| | Aib-TFI-(D-Glu)-YLIEGGPSSGAPPPS- | | | | |
| | NH₂ | | | | |
| 171 | Y-Aib-EGT-αMeF(2F)-TSDHSI-αMeL- | 82 | 0.471 | 0.0609 | 7.73 |
| | LD-Orn-K((2-[2-(2-Amino-ethoxy)- | | | | |
| | ethoxy]-acetyl)₂-(γGlu)- | | | | |
| | CO—(CH₂)₁₈—CO₂H)AQ-Aib-TFI-(D- | | | | |
| | Glu)-YLIEGGPSSGAPPPS-NH₂ | | | | |
| 172 | Y-Aib-EGT-αMeF(2F)-TSDHSI-αMeL- | 83 | 0.1 | 0.038 | 2.63 |
| | LD-Dab-K((2-[2-(2-Amino-ethoxy)- | | | | |
| | ethoxy]-acetyl)₂-(γGlu)- | | | | |
| | CO—(CH₂)₁₈—CO₂H)AQ-Aib-TFI-(D- | | | | |
| | Glu)-YLIEGGPSSGAPPPS-NH₂ | | | | |
| 173 | Y-Aib-EGT-αMeF(2F)-TSDHSI-αMeL- | 84 | 0.179 | 0.0373 | 4.80 |
| | LD-Dap-K((2-[2-(2-Amino-ethoxy)- | | | | |
| | ethoxy]-acetyl)₂-(γGlu)- | | | | |
| | CO—(CH₂)₁₈—CO₂H)AQ-Aib-TFI-(D- | | | | |
| | Glu)-YLIEGGPSSGAPPPS-NH₂ | | | | |
| 174 | Y-Aib-EGT-αMeF(2F)-TSDHSI-αMeL- | 85 | 0.483 | 0.0968 | 4.99 |
| | LDEK((2-[2-(2-Amino-ethoxy)-ethoxy]- | | | | |
| | acetyl)₂-CO—(CH₂)₁₈—CO₂H)AQ-Aib- | | | | |
| | TFI-(D-Glu)-YLIEGGPSSGAPPPS-NH₂ | | | | |
| 176 | Y-Aib-EGT-αMeF(2F)-TSDHSI-αMeL- | 86 | 0.201 | 0.0427 | 4.71 |
| | LDEK((2-[2-(2-Amino-ethoxy)-ethoxy]- | | | | |
| | acetyl)₂-(εK)-CO—(CH₂)₁₆—CO₂H)AQ- | | | | |
| | Aib-TFI-(D-Glu)-YLIEGGPSSGAPPPS- | | | | |
| | NH₂ | | | | |
| 178 | Y-Aib-EGT-αMeF(2F)-TSDHSI-αMeL- | 87 | 0.0341 | 0.00349 | 9.77 |
| | LDEK((2-[2-(2-Amino-ethoxy)-ethoxy]- | | | | |
| | acetyl)₂-(γGlu)-CO—(CH₂)₁₄—CO₂H)AQ- | | | | |
| | Aib-HFI-(D-Glu)- | | | | |
| | YLIEGGPSSGAPPPS-NH₂ | | | | |
| 179 | Y-Aib-EGT-αMeF(2F)-TSDHSI-αMeL- | 88 | 0.0575 | 0.0169 | 3.40 |
| | LDEK((2-[2-(2-Amino-ethoxy)-ethoxy]- | | | | |
| | acetyl)₂-(γGlu)-CO—(CH₂)₁₆—CO₂H)AQ- | | | | |
| | Aib-HFI-(D-Glu)-YLIEGGPSSG-NH₂ | | | | |
| 180 | Y-Aib-EGT-αMeF(2F)-TSDHSI-αMeL- | 89 | 0.133 | 0.0212 | 6.27 |
| | LDEK((2-[2-(2-Amino-ethoxy)-ethoxy]- | | | | |
| | acetyl)₂-(γGlu)-CO—(CH₂)₁₆—CO₂H)AQ- | | | | |
| | Aib-HFI-(D-Glu)-YLIEGG-NH₂ | | | | |
| 181 | Y-Aib-EGT-αMeF(2F)-TSD-3Pal-SI- | 90 | 4.1 | 0.718 | 5.71 |
| | αMeL-LDEK((2-[2-(2-Amino-ethoxy)- | | | | |
| | ethoxy]-acetyl)₂-(γGlu)- | | | | |
| | CO—(CH₂)₁₈—CO₂H)AQ-Aib-EFI-(D- | | | | |
| | Glu)-YLIEGGPSSGAPPPS-NH₂ | | | | |
| 182 | Y-Aib-EGT-αMeF(2F)-TSD-4Pal-SI- | 91 | 4.37 | 0.873 | 5.01 |
| | αMeL-LDEK((2-[2-(2-Amino-ethoxy)- | | | | |
| | ethoxy]-acetyl)₂-(γGlu)- | | | | |
| | CO—(CH₂)₁₈—CO₂H)AQ-Aib-EFI-(D- | | | | |
| | Glu)-YLIEGGPSSGAPPPS-NH₂ | | | | |
| 183 | Y-Aib-EGT-αMeF(2F)-TSDLSI-αMeL- | 92 | 3.1 | 0.843 | 3.68 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)-YLIEGGPSSGAPPPS- | | | | |
| | NH₂ | | | | |
| 187 | Y-Aib-EGT-αMeF(2F)-TSDQSI-αMeL- | 93 | 4.61 | 0.702 | 6.57 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)-YLIEGGPSSGAPPPS- | | | | |
| | NH₂ | | | | |
| 189 | Y-Aib-EGT-αMeF(2F)-TSD-4Pal-SI- | 94 | 2.41 | 0.668 | 3.61 |
| | αMeL-LDEK((2-[2-(2-Amino-ethoxy)- | | | | |
| | ethoxy]-acetyl)₂-(γGlu)- | | | | |
| | CO—(CH₂)₁₈—CO₂H)AQ-Aib-EFI-Aib- | | | | |
| | YLIEGGPSSGAPPPS-NH₂ | | | | |
| 197 | Y-αMePro-EGTFTSDYSILLDEK((2-[2- | 95 | 0.855 | 0.189 | 4.52 |
| | (2-Amino-ethoxy)-ethoxy]-acetyl)₂- | | | | |
| | (γGlu)-CO—(CH₂)₁₆—CO₂H) | | | | |
| | AQQAFIEYLIEGGPSSG-NH₂ | | | | |
| 202 | Y-Aib-EGT-αMeF(2F)-TSD-4Pal-SI- | 96 | 4.79 | 0.712 | 6.73 |
| | αMeL-LDKK((2-[2-(2-Amino-ethoxy)- | | | | |
| | ethoxy]-acetyl)-(γGlu)-(2-[2-(2-Amino- | | | | |
| | ethoxy)-ethoxy]-acetyl)- | | | | |
| | CO—(CH₂)₁₈—CO₂H)AQ-Aib-EFI-(D- | | | | |
| | Glu)-YLIEGGPSSGAPPPS-NH₂ | | | | |
| 203 | Y-Aib-EGT-αMeF(2F)-TSD-4Pal-SI- | 97 | 4.95 | 0.671 | 7.38 |
| | αMeL-LDKK((2-[2-(2-Amino-ethoxy)- | | | | |
| | ethoxy]-acetyl)₂-(γGlu)- | | | | |
| | CO—(CH₂)₁₈—CO₂H)AQ-Aib-EFI-(D- | | | | |
| | Glu)-YLIEGGPSSGAPPPS-NH₂ | | | | |
| 204 | Y-Aib-EGT-αMeF(2F)-TSD-4Pal-SI- | 98 | 4.58 | 0.689 | 6.65 |
| | αMeL-LDEK((2-[2-(2-Amino-ethoxy)- | | | | |
| | ethoxy]-acetyl)₂-(γGlu)- | | | | |
| | CO—(CH₂)₁₆—CO₂H)AQ-Aib-EFI-(D- | | | | |
| | Glu)-YLIEGGPSSGAPPPS-NH₂ | | | | |
| 205 | Y-Aib-EGT-αMeF(2F)-TSD-4Pal-SI- | 99 | 5.71 | 1.46 | 3.91 |
| | αMeL-LDEK((2-[2-(2-Amino-ethoxy)- | | | | |
| | ethoxy]-acetyl)₂-CO—(CH₂)₁₆—CO₂H)AQ- | | | | |
| | Aib-EFI-(D-Glu)-YLIEGGPSSGAPPPS- | | | | |
| | NH₂ | | | | |
| 206 | Y-Aib-EGT-αMeF(2F)-TSD-4Pal-SI- | 100 | 4.75 | 1.42 | 3.35 |
| | αMeL-LDEK((2-[2-(2-Amino-ethoxy)- | | | | |
| | ethoxy]-acetyl)₂CO—(CH₂)₁₈—CO₂H)AQ- | | | | |
| | Aib-EFI-(D-Glu)-YLIEGGPSSGAPPPS- | | | | |
| | NH₂ | | | | |
| 207 | Y-Aib-EGT-αMeF(2F)-TSD-4Pal-SI- | 101 | 4.76 | 1.23 | 3.87 |
| | αMeL-LDEK((2-[2-(2-Amino-ethoxy)- | | | | |
| | ethoxy]-acetyl)₂-(εK)- | | | | |
| | CO—(CH₂)₁₈—CO₂H)AQ-Aib-EFI-(D- | | | | |
| | Glu)-YLIEGGPSSGAPPPS-NH₂ | | | | |
| 208 | Y-Aib-EGT-αMeF(2F)-TSDYSI-αMeL- | 102 | 5.33 | 0.587 | 9.08 |
| | LDEK((2-[2-(2-Amino-ethoxy)-ethoxy]- | | | | |
| | acetyl)₂-(γGlu)-CO—(CH₂)₁₄—CO₂H)AQ- | | | | |
| | Aib-EFI-(D-Glu)-YLIEGGPSSGAPPPS- | | | | |
| | NH₂ | | | | |
| 209 | Y-Aib-EGT-αMeF(2F)-TSDYSI-αMeL- | 203 | 5.73 | 1.12 | 5.12 |
| | LD-Dab-K((2-[2-(2-Amino-ethoxy)- | | | | |
| | ethoxy]-acetyl)₂-(γGlu)- | | | | |
| | CO—(CH₂)₁₆—CO₂H)AQ-Aib-EFI-(D- | | | | |
| | Glu)-YLIEGGPSSGAPPPS-NH₂ | | | | |
| 210 | Y-Aib-EGT-αMeF(2F)-TSDYSI-αMeL- | 104 | 5.4 | 1.03 | 5.24 |
| | LD-Dap-K((2-[2-(2-Amino-ethoxy)- | | | | |
| | ethoxy]-acetyl)₂-(γGlu)- | | | | |
| | CO—(CH₂)₁₆—CO₂H)AQ-Aib-EFI-(D- | | | | |
| | Glu)-YLIEGGPSSGAPPPS-NH₂ | | | | |
| 211 | Y-Aib-EGT-αMeF(2F)-TSDYSI-αMeL- | 105 | 4.59 | 1.28 | 3.59 |
| | LDKK((2-[2-(2-Amino-ethoxy)-ethoxy]- | | | | |
| | acetyl)-(γGlu)-(2-[2-(2-Amino-ethoxy)- | | | | |
| | ethoxy]-acetyl)-CO—(CH₂)₁₈—CO₂H)AQ- | | | | |
| | Aib-EFI-(D-Glu)-YLIEGGPSSGAPPPS- | | | | |
| | NH₂ | | | | |
| 212 | Y-Aib-EGT-αMeF(2F)-TSDYSI-αMeL- | 106 | 4.17 | 0.771 | 5.41 |
| | LDKK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)-YLIEGGPSSGAPPPS- | | | | |
| | NH₂ | | | | |
| 213 | Y-Aib-EGT-αMeF-TSDYSI-αMeL- | 107 | 3.87 | 0.694 | 5.58 |
| | LDEK((2-[2-(2-Amino-ethoxy)-ethoxy]- | | | | |
| | acetyl)₂-(γGlu)-CO—(CH₂)₁₆—CO₂H)AQ- | | | | |
| | Aib-EFI-(D-Glu)-YLIEGGPSSGAPPPS- | | | | |
| | NH₂ | | | | |
| 214 | Y-Aib-EGT-αMeF(2F)-TSDYSI-αMeL- | 108 | 6.92 | 1.74 | 3.98 |
| | LDEK((2-[2-(2-Amino-ethoxy)- | | | | |
| | ethoxy]-acetyl)₂-CO—(CH₂)₁₆—CO₂H)AQ- | | | | |
| | Aib-EFI-(D-Glu)-YLIEGGPSSGAPPPS- | | | | |
| | NH₂ | | | | |
| 215 | Y-Aib-EGT-αMeF(2F)-TSDYSI-αMeL- | 109 | 3.53 | 0.813 | 4.34 |
| | LDEK((2-[2-(2-Amino-ethoxy)-ethoxy]- | | | | |
| | acetyl)₂-CO—(CH₂)₁₈—CO₂H)AQ-Aib- | | | | |
| | EFI-(D-Glu)-YLIEGGPSSGAPPPS-NH₂ | | | | |
| 216 | Y-Aib-EGT-αMeF(2F)-TSDYSI-αMeL- | 110 | 4.91 | 1.31 | 3.75 |
| | LDEK((2-[2-(2-Amino-ethoxy)-ethoxy]- | | | | |
| | acetyl)₂-(εK)-CO—(CH₂)₁₈—CO₂H)AQ- | | | | |
| | Aib-EFI-(D-Glu)-YLIEGGPSSGAPPPS- | | | | |
| | NH₂ | | | | |
| 217 | Y-Aib-EGT-αMeF(2F)-TSDYSI-αMeL- | 111 | 3.41 | 1.14 | 2.99 |
| | LD-Dab-K((2-[2-(2-Amino-ethoxy)- | | | | |
| | ethoxy]-acetyl)₂-(γGlu)- | | | | |
| | CO—(CH₂)₁₈—CO₂H)AQ-Aib-EFI-(D- | | | | |
| | Glu)-YLIEGGPSSGAPPPS-NH₂ | | | | |
| 218 | Y-Aib-EGT-αMeF(2F)-TSDYSI-αMeL- | 112 | 4.43 | 1.02 | 4.34 |
| | LD-Dap-K((2-[2-(2-Amino-ethoxy)- | | | | |
| | ethoxy]-acetyl)₂-(γGlu)- | | | | |
| | CO—(CH₂)₁₈—CO₂H)AQ-Aib-EFI-(D- | | | | |
| | Glu)-YLIEGGPSSGAPPPS-NH₂ | | | | |
| 219 | Y-Aib-EGT-αMeF(2F)-TSDYSI-αMeL- | 113 | 5.86 | 1.03 | 5.69 |
| | LDEK((2-[2-(2-Amino-ethoxy)-ethoxy]- | | | | |
| | acetyl)₂-(γGlu)-CO—(CH₂)₁₆—CO₂H)AQ- | | | | |
| | Aib-EFI-(D-Glu)-YLIEGGPSSG-NH₂ | | | | |
| 220 | Y-Aib-EGT-αMeF(2F)-TSDYSI-αMeL- | 114 | 6.3 | 1.36 | 4.63 |
| | LDEK((2-[2-(2-Amino-ethoxy)-ethoxy]- | | | | |
| | acetyl)₂-(γGlu)-CO—(CH₂)₁₆—CO₂H)AQ- | | | | |
| | Aib-EFI-(D-Glu)-YLIEGG-NH₂ | | | | |
| 221 | Y-Aib-EGT-αMeF(2F)-TSDYSI-αMeL- | 115 | 4.5 | 0.795 | 5.66 |
| | LDEK((2-[2-(2-Amino-ethoxy)-ethoxy]- | | | | |
| | acetyl)₂-(γGlu)-CO—(CH₂)₁₆—CO₂H)AQ- | | | | |
| | Aib-EFI-(D-Glu)-YLIEGGPSSGAPPPS- | | | | |
| | NH₂ | | | | |
| 222 | Y-Aib-EGT-αMeF(2F)-TSDYSI-αMeL- | 116 | 5.84 | 1.55 | 3.77 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)--αMeY- | | | | |
| | LIEGGPSSGAPPPS-NH₂ | | | | |
| 223 | Y-Aib-EGT-αMeF(2F)-TSDYSI-αMeL- | 117 | 2.93 | 0.962 | 3.05 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFIE-αMeY-LIEGGPSSGAPPPS- | | | | |
| | NH₂ | | | | |
| 224 | Y-Aib-EGT-αMeF(2F)-TSDYSI-Aib- | 118 | 3.52 | 1.06 | 3.32 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)-YLIEGGPSSGAPPPS- | | | | |
| | NH₂ | | | | |
| 225 | Y-Aib-EGT-αMeF-TSDYSI-Aib- | 119 | 1.99 | 0.523 | 3.80 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)-YLIEGGPSSGAPPPS- | | | | |
| | NH₂ | | | | |
| 226 | Y-Aib-EGT-αMeF(2F)- | 120 | 4.27 | 1.25 | 3.42 |
| | TSDYSILLDEK((2-[2-(2-Amino- | | | | |
| | ethoxy)-ethoxy]-acetyl)₂-(γGlu)- | | | | |
| | CO—(CH₂)₁₈—CO₂H)AQ-Aib-EFI-(D- | | | | |
| | Glu)-YLIEGGPSSGAPPPS-NH₂ | | | | |
| 227 | Y-Aib-EGT-αMeF-TSDYSI-αMeL- | 121 | 4.73 | 1.07 | 4.42 |
| | LDEK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)-YLIEGGPSSGAPPPS- | | | | |
| | NH₂ | | | | |
| 228 | Y-Aib-EGT-αMeF-TSDYSI-αMeL- | 122 | 3.86 | 1.1 | 3.51 |
| | LDKK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)-YLIEGGPSSG-NH₂ | | | | |
| 230 | Y-Aib-EGT-αMeF(2F)-TSDYSI-αMeL- | 123 | 3.31 | 0.599 | 5.53 |
| | LDHK((2-[2-(2-Amino-ethoxy)-ethoxy]- | | | | |
| | acetyl)₂-(γGlu)-CO—(CH₂)₁₈—CO₂H)AQ- | | | | |
| | Aib-EFI-(D-Glu)-YLIEGGPSSGAPPPS- | | | | |
| | NH₂ | | | | |
| 233 | Y-Aib-EGT-αMeF(2F)-TSD-4Pal-SI- | 124 | 4.95 | 0.535 | 9.25 |
| | αMeL-LD-Dab-K((2-[2-(2-Amino- | | | | |
| | ethoxy)-ethoxy]-acetyl)₂-(γGlu)- | | | | |
| | CO—(CH₂)₁₆—CO₂H)AQ-Aib-EFI-(D- | | | | |
| | Glu)-YLIEGGPSSGAPPPS-NH₂ | | | | |
| 236 | Y-Aib-EGT-αMeF(2F)-TSDYSI-αMeL- | 125 | 6.76 | 1.65 | 4.10 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-EFI (D- | | | | |
| | Glu)-αMeY-LIEGGPSSGAPPPS-NH₂ | | | | |
| 238 | Y-Aib-EGT-αMeF(2F)-TSDLSI-αMeL- | 126 | 7.33 | 1.15 | 6.37 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-EFI-(D- | | | | |
| | Glu)-αMeY-LIEGGPSSGAPPPS-NH₂ | | | | |
| 239 | Y-Aib-EGT-αMeF(2F)-TSD-4Pal-SI- | 127 | 5.27 | 0.987 | 5.34 |
| | αMeL-LD-Orn-K((2-[2-(2-Amino- | | | | |
| | ethoxy)-ethoxy]-acetyl)₂-(γGlu)- | | | | |
| | CO—(CH₂)₁₆—CO₂H)AQ-Aib-TFI-(D- | | | | |
| | Glu)-αMeY-LIEGGPSSGAPPPS-NH₂ | | | | |
| 241 | Y-Aib-EGT-αMeF(2F)-TSDVSI-αMeL- | 128 | 7.12 | 1.89 | 3.77 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-TFI-(D- | | | | |
| | Glu)-αMeY-LIEGGPSSGAPPPS-NH₂ | | | | |
| 242 | Y-Aib-EGT-αMeF(2F)-TSDVSI-αMeL- | 129 | 5.58 | 1.96 | 2.85 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-AFI-(D- | | | | |
| | Glu)-αMeY-LIEGGPSSGAPPPS-NH₂ | | | | |
| 243 | Y-Aib-EGT-αMeF(2F)-TSDLSI-αMeL- | 130 | 8.69 | 1.22 | 7.12 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-TFI-(D- | | | | |
| | Glu)-αMeY-LIEGGPSSGAPPPS-NH₂ | | | | |
| 244 | Y-Aib-EGT-αMeF(2F)-TSDLSI-αMeL- | 131 | 8.27 | 1.29 | 6.41 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-AFI-(D- | | | | |
| | Glu)-αMeY-LIEGGPSSGAPPPS-NH₂ | | | | |
| 246 | Y-Aib-EGT-αMeF(2F)-TSDASI-αMeL- | 132 | 6.36 | 2.56 | 2.48 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-AFI-(D- | | | | |
| | Glu)-αMeY-LIEGGPSSGAPPPS-NH₂ | | | | |
| 247 | Y-Aib-EGT-αMeF(2F)-TSDYSI-Aib- | 133 | 2.58 | 0.614 | 4.20 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-EFI-(D- | | | | |
| | Glu)-YLIEGGPSSGAPPPS-NH₂ | | | | |
| 248 | Y-Aib-EGT-αMeF(2F)-TSDYSILLD- | 134 | 5.08 | 0.8 | 6.35 |
| | Orn-K((2-[2-(2-Amino-ethoxy)-ethoxy]- | | | | |
| | acetyl)₂-(γGlu)-CO—(CH₂)₁₆—CO₂H)AQ- | | | | |
| | Aib-EFI-(D-Glu)-YLIEGGPSSGAPPPS- | | | | |
| | NH₂ | | | | |
| 249 | Y-Aib-EGT-αMeF(2F)-TSDYSI-Nle- | 135 | 3.84 | 0.778 | 4.94 |
| | LD-Orn-K((2-[2-(2-Amino-ethoxy)- | | | | |
| | ethoxy]-acetyl)₂-(γGlu)- | | | | |
| | CO—(CH₂)₁₆—CO₂H)AQ-Aib-EFI-(D- | | | | |
| | Glu)-YLIEGGPSSGAPPPS-NH₂ | | | | |
| 250 | Y-Aib-EGT-αMeF(2F)-TSDYSI-Aib- | 136 | 4.27 | 0.985 | 4.34 |
| | LD-Orn-K((2-[2-(2-Amino-ethoxy)- | | | | |
| | ethoxy]-acetyl)₂-(γGlu)- | | | | |
| | CO—(CH₂)₁₈—CO₂H)AQ-Aib-EFI-(D- | | | | |
| | Glu)-YLIEGGPSSGAPPPS-NH₂ | | | | |
| 251 | Y-Aib-EGT-αMeF(2F)-TSD-4Pal-SI- | 137 | 5.12 | 1.37 | 3.74 |
| | αMeL-LD-Orn-K((2-[2-(2-Amino- | | | | |
| | ethoxy)-ethoxy]-acetyl)₂-(εK)- | | | | |
| | CO—(CH₂)₁₈—CO₂H)AQ-Aib-AFI-(D- | | | | |
| | Glu)-αMeY-LIEGGPSSGAPPPS-NH₂ | | | | |
| 264 | Y-Aib-EGT-αMeF(2F)-TSD-4Pal-SI- | 138 | 8.4 | 3.19 | 2.63 |
| | αMeL-LD-Orn-K((2-[2-(2-Amino- | | | | |
| | ethoxy)-ethoxy]-acetyl)₂-(γGlu)- | | | | |
| | CO—(CH₂)₁₆—CO₂H)AQ-Aib-EFI-(D- | | | | |
| | Glu)-αMeY-LIEGGPSSG-NH₂ | | | | |
| 265 | Y-Aib-EGT-αMeF(2F)-TSD-4Pal-SI- | 139 | 9.77 | 3.27 | 2.99 |
| | αMeL-LD-Orn-K((2-[2-(2-Amino- | | | | |
| | ethoxy)-ethoxy]-acetyl)₂-(γGlu)- | | | | |
| | CO—(CH₂)₁₆—CO₂H)AQ-Aib-EFI-(D- | | | | |
| | Glu)-αMeY-LIEGG-NH₂ | | | | |

As demonstrated by data in Table 1, the peptides, normalized to baseline and native peptides, stimulate cAMP from human GLP-1R and GIPR in the presence of 0.1% casein with a GIP potency ratio that is about 2.5 to about 10 times the GLP-1 receptor potency.

In an embodiment, is a GIP: GLP-1 coagonist compound wherein the peptide is a potent GIPR/GLP-1R dual agonist that is a partial agonist on the GLP-1R as shown by Cell Membrane Guanosine 5′-(gamma-thio) Triphosphate-[35S] (GTPγS) Binding Assay, and a partial agonist on the GLP-1R as shown by the β-arrestin-2 recruitment assay. An embodiment, is a GIP: GLP-1 co-agonist compound, or pharmaceutically acceptable salt thereof, wherein the compound stimulates GLP-1R induced activation of Gas in the GLP-1R HEK293 Cell Membrane Guanosine 5′-(gamma-thio) Triphosphate-[35S] (GTPγS) Binding Assay. In an embodiment, is a compound showing partial agonism of 75% or less in the GLP-1R HEK293 Cell Membrane Guanosine 5′-(gamma-thio) Triphosphate-[35S] (GTPγS)

Binding Assay, and 35% or less in the GLP-CHO Cell β-Arrestin.Recruitment Assay. In an embodiment is a method for treating diabetes comprising administering an effective amount of a compound showing partial agonism of 75% or less in the GLP-1R HEK293 Cell Membrane Guanosine 5′-(gamma-thio) Triphosphate-[35S] (GTPγS) Binding Assay, and an effective amount of a compound that is a GIP agonist. In an embodiment, the compound showing partial agonism in the GLP-1R HEK293 Cell

Membrane Guanosine 5′-(gamma-thio) Triphosphate-[35S] (GTPγS) Binding Assay is co-administered with a compound having GIP agonist activity. In an embodiment, the compound showing partial agonism in the GLP-1R HEK293 Cell Membrane Guanosine 5′-(gamma-thio) Triphosphate-[35S] (GTPγS) Binding Assay is administered as an active agent within one week before or after a compound having GIP agonist activity. In an embodiment, a method for treating diabetes comprises administering an effective amount of a compound showing 35% or less in the GLP-CHO Cell β-Arrestin.Recruitment Assay and administering an effective amount of a compound showing partial agonism of 75% or less in the GLP-1R HEK293 Cell Membrane Guanosine 5′-(gamma-thio) Triphosphate-[35S] (GTPγS) Binding Assay.

### GLP-1R HEK293 Cell Membrane [³⁵S] GTPγS Binding Assay

The GLP-1 receptor is a G-protein coupled receptor that increases GTP-bound Gαₛ upon ligand induced receptor activation. The potency of peptides to stimulate-GLP-1R induced activation of Gαₛ is determined using preparations of purified membranes from HEK293 cells expressing the human GLP-1R. The assay is performed similarly to that as previously described (Bueno et al., J. Biol. Chem., (2016) 291, 10700 and Willard et al., Mol. Pharmacol. (2012) 82,1066). The test peptides are solubilized in DMSO and diluted in reaction buffer containing 5 μg of membrane in 20 mM HEPES pH 7.4, 50 mM NaCl, 5 mM MgCl₂, 40 μg/ml saponin, 0.1% BSA, and 500 μM 35S-labeled GTPγS for 30 minutes at room temperature. Reactions are terminated by addition of 0.2% Nonidet P-40 detergent containing rabbit anti-Gas polyclonal antibody and 0.5 mg of anti-rabbit polyvinyltoluene beads. Mixtures are developed for 30 minutes, centrifuged at 80×g for 10 minutes, and counted for 1 minute/well using a MicroBeta TriLux instrument. Peptide concentration-response curves are fit to a four-parameter logistic model to calculate potency as an EC₅₀. Data normalization to % stimulation is performed using DMSO and GLP-1 (7-36) as minimum and maximum controls for the receptor (Campbell et al, Assay Guidance Manual 2017). The potency of a sample peptide to stimulate GIPR induced activation of Gαₛ is determined. Assay results identify a peptide that is a partial agonist on the GLP-1R with respect to GLP-1R induced activation of Gas.

### GLP-1R CHO Cell β-Arrestin Recruitment Assay

Activated G-protein coupled receptors can interact with the β-arrestin family of signalling proteins. The potency of peptides for GLP-1R induced arrestin recruitment is determined using the PathHunter Enzyme Fragment Complementation approach substantially as described (von Degenfeld et al., FASEB J., 2007 (14): 3819-26 and Hamdouchi et al., J. Med Chem., 2016 59 (24): 10891-10916). CHO-K1 cells expressing Pro-Link-tagged Human GLP-1R and enzyme-acceptor-tagged β-arrestin-2 may be obtained from DiscoveRx and prepared as assay-ready frozen cells. Test peptides are solubilized in DMSO and serial dilutions are perfomed using the Echo acoustic dispenser (LabCyte). Assay media is the PathHunter Cell Assay Buffer (DiscoveRx) containing 0.1% w/v hydrolyzed Casein (Sigma). 100 nl of peptide is dispensed into 10 μl of assay media in a 384 well plate and then 10 μl of cells in assay media are added to give 5000 cells per well. Plates are incubated for 90 minutes in a 37′C/5% C02 incubator and 10 μl of PathHunter detection reagent is added (DiscoveRx) and plates are incubated at room temperature for 60 minutes. Luminescence signal is measured. Peptide concentration-response curves fit to a four-parameter logistic model to calculate potency as an EC50. Data normalization to % stimulation is performed using DMSO and GLP-1 (7-36) as minimum and maximum controls (Campbell et al, Assay Guidance Manual 2017). The potency of a sample peptide to stimulate GLP-1R induced β-arrestin recruitment is determined. Assay results identify a peptide that is a partial agonist on the GLP-1R with respect to β-arrestin-2 recruitment.

### Clinical Study to Determine GIP: GLP-1 Peptide Maintenance Dose

A 6-month (26-week) Phase 2 double-blind clinical study is designed to evaluate the safety, efficacy, and PK/PD of 4 dose levels (1 mg, 5 mg, 10 mg and 15 mg respectively) of a GIP: GLP-1 Peptide administered once weekly by subcutaneous injection compared with dulaglutide 1.5 mg administered once weekly (QW) and placebo QW in patients with T2DM who have inadequate glycemic control with diet and exercise with or without a stable dose of metformin. The GIP: GLP Peptide dose was up-titrated to the maintenance dose using the following weekly dose increments:

| GIP:GLP-1 | Weekly GIP:GLP-1 |
|---|---|
| Peptide dose: | Peptide Dose Increments: |
| 1 mg | Week 0-26: 1 mg QW |
| 5 mg | Week 0-26: 5 mg QW |
| 10 mg | Week 0: 5 mg |
| | Week 1: 5 mg |
| | Week 2-26: 10 mg |
| 15 mg | Week 0: 5 mg |
| | Week 1: 5 mg |
| | Week 2: 10 mg |
| | Week 3: 10 mg |
| | Week 4: 10 mg |
| | Week 5: 10 mg |
| | Week 6-26: 15 mg |

The study also has a 4-week follow up period. In addition to safety and efficacy for treating T2DM, efficacy endpoints include the effect of the GIP: GLP-1 Peptide on HbA1c, FBG, body weight, lipids, and waist circumference compared with placebo and with dulaglutide 1.5 mg. The study also evaluates the effect of the GIP: GLP-1 Peptide on GI tolerability, hypoglycemia, hypersensitivity reactions, and pancreatic safety, as well as the development of treatment-emergent anti-drug antibodies. Model-based dose response analyses are performed to predict potential for significant HbA1c lowering and weight loss in longer studies.

### Statistical Analyses:

Efficacy: The primary efficacy outcome of HbA1c change from baseline to the 26-week endpoint is analyzed using a Bayesian dose-response model. Analyses are performed on the intention to treat population (mITT) analysis set. Supportive analysis of the primary efficacy outcome for the mITT dataset are the model for post-baseline measures (MMRM) with body mass index (BMI) (<30 kg/m², ≥30 kg/m²), metformin use, treatment, visit, and treatment-by-visit interaction as fixed effects, baseline HbA1c as a covariate, and patient as a random effect.

The mean weight change from baseline at 12 and 26 weeks, along with the mean change from baseline of HbA1c at 12 weeks, is analyzed using similar dose-response models as the primary analyses. The percentages of patients with ≥5% or ≥10% body weight loss, reaching the HbA1c target of ≤6.5% or ≤7.0% at 26 weeks, or requiring rescue therapy are analyzed using logistic regression with fixed effects of treatment and strata, and baseline as a covariate. The changes from baseline in FBG (fasting blood glucose), SMBG (self-monitored blood glucose) levels, waist circumference, and mean percentage change in lipids from baseline to 12 and 26 weeks are analyzed using a similar MMRM to the one used for the primary analyses.

### Clinical Study to Determine GIP: GLP-1 Peptide Titration Schedule

This is a 12-week treatment with a 1 week screening (Visit 1) followed by a 1 week lead-in (Visit 2), then 12 weeks of treatment (Visits 3-10, including telephone visits), then followed by 4-week safety follow-up. It is a Phase 2 study designed to examine the efficacy and tolerability of subcutaneously once-weekly administration of a GIP: GLP-1 Peptide compared with placebo in patients with type 2 diabetes who have inadequate glycemic control with diet and exercise alone or with a stable dose of metformin. The study was designed per below and conducted to refine the titration scheme.

| GIP:GLP-1 | Weekly GIP:GLP-1 |
|---|---|
| Peptide Dose: | Peptide Dose Increments: |
| Placebo | Week 1-12 |
| Group 1 | Weeks 1-2: 2.5 mg |
| | Weeks 3-4: 5 mg |
| | Weeks 5-8: 10 mg |
| | Weeks 9-12: 15 mg |
| Group 2 | Weeks 1-4: 2.5 mg |
| | Weeks 5-8: 7.5 mg |
| | Weeks 9-12: 15 mg |
| Group 3 | Weeks 1-4: 4 mg |
| | Weeks: 5-8: 8 mg |
| | Weeks 9-12: 12 mg |

Amino Acid Sequences
GIP (Human)
SEQ ID NO: 1
YAEGTFISDYSIAMDKIHQQDFVNWLLAQKGKKNDWKHNITQ
GLP-1 (7-36) (Human)
SEQ ID NO: 2
HAEGTFTSDVSSYLEGQAAKEFIAWLVKGR-NH₂
SEQ ID NO: 3
R₁X₁X₂X₃GTX₆TSDX₁₀X₁₁X₁₂X₁₃X₁₄DX₁₆X₁₇AX₁₉
X₂₀X₂₁X₂₂X₂₃X₂₄X₂₅X₂₆X₂₇X₂₈X₂₉X₃₀X₃₁

We claim:

1-16. (canceled)

17. A method of treating NASH in a patient in need thereof, comprising:
a) administering to said patient a first titration dose of a GIP: GLP-1 Peptide having a GIP: GLP-1 receptor agonist potency ratio that is greater than or equal to the GIP: GLP-1 receptor agonist potency ratio of a GIP: GLP-1 Peptide having SEQ ID NO: 56 and less than the GIP: GLP-1 receptor agonist potency ratio of a GIP: GLP-1 Peptide having SEQ ID NO: 49, wherein the GIP: GLP-1 receptor agonist potency ratio is measured after a 60 minute incubation using a casein cAMP assay normalized against GIP and GLP-1 ; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
wherein the first titration dose is about 25% of the maintenance dose, the second titration dose is about 50% of the maintenance dose and the third titration dose is about 75% of the maintenance dose.

18-21. (canceled)

22. The method of claim 17, wherein the doses are each administered for about four weeks before the administration of the next higher dose begins.

23. (canceled)

24. The method of claim 17, wherein the GIP: GLP-1 Peptide has a receptor agonist potency ratio that is 2.5:1 to about 5:1 GIP to GLP-1.

25. The method of claim 17, wherein the GIP: GLP-1 Peptide has a receptor agonist potency ratio that is 2.5:1 to about 3.5:1 GIP to GLP-1.

26-31. (canceled)

1. A method of treating type 2 diabetes in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
wherein the titration dose is about 50% of the maintenance dose.

2. A method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
wherein the first titration dose is about 25% of the maintenance dose, the second titration dose is about 50% of the maintenance dose and the third titration dose is about 75% of the maintenance dose.

3. A method of treating type 2 diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

4. A method of improving glycemic control in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
wherein the titration dose is about 50% of the maintenance dose.

5. A method of improving glycemic control in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
wherein the first titration dose is about 25% of the maintenance dose, the second titration dose is about 50% of the maintenance dose and the third titration dose is about 75% of the maintenance dose.

6. A method of improving glycemic control in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

7. A method of improving weight management in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
wherein the titration dose is about 50% of the maintenance dose.

8. A method of improving weight management in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
wherein the first titration dose is about 25% of the maintenance dose, the second titration dose is about 50% of the maintenance dose and the third titration dose is about 75% of the maintenance dose.

9. A method of improving weight management in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

10. A method of treating chronic kidney disease in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
wherein the titration dose is about 50% of the maintenance dose.

11. A method of treating chronic kidney disease in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
wherein the first titration dose is about 25% of the maintenance dose, the second titration dose is about 50% of the maintenance dose and the third titration dose is about 75% of the maintenance dose.

12. A method of treating chronic kidney disease in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

13. A method of treating NAFLD in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
wherein the titration dose is about 50% of the maintenance dose.

14. A method of treating NAFLD in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
wherein the first titration dose is about 25% of the maintenance dose, the second titration dose is about 50% of the maintenance dose and the third titration dose is about 75% of the maintenance dose.

15. A method of treating NAFLD in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

16. A method of treating NASH in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
wherein the titration dose is about 50% of the maintenance dose.

17. A method of treating NASH in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
wherein the first titration dose is about 25% of the maintenance dose, the second titration dose is about 50% of the maintenance dose and the third titration dose is about 75% of the maintenance dose.

18. A method of treating NASH in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

19. A method of curing diabetes, inducing remission or regression of diabetes, or preventing diabetes in a patient in need thereof, comprising:
a) administering to said patient a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a maintenance dose of that GIP: GLP-1 Peptide;
wherein the titration dose is about 50% of the maintenance dose.

20. A method of curing diabetes, inducing remission or regression of diabetes, or preventing diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a maintenance dose of that GIP: GLP-1 Peptide;
wherein the first titration dose is about 25% of the maintenance dose, the second titration dose is about 50% of the maintenance dose and the third titration dose is about 75% of the maintenance dose.

21. A method of curing diabetes, inducing remission or regression of diabetes, or preventing diabetes in a patient in need thereof, comprising:
a) administering to said patient a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administering to said patient a second titration dose of that GIP: GLP-1 Peptide for a minimum of about two weeks; and thereafter
c) administering to said patient a third titration dose of that GIP: GLP-1 Peptide for a minimum of about two weeks; and thereafter
d) administering to said patient a fourth titration dose of that GIP: GLP-1 Peptide for a minimum of about two weeks; and thereafter
e) administering to said patient a fifth titration dose of that GIP: GLP-1 Peptide for a minimum of about two weeks; and thereafter
f) administering to said patient a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

22. The method of any one of claims 1-21, wherein the titration doses are each administered for about four weeks before the administration of the next higher dose begins.

23. The method of any one of claims 1-22, wherein the GIP: GLP-1 Peptide is a compound of SEQ ID NO: 3, or a pharmaceutically acceptable salt thereof.

24. The method of any one of claims 1-23, wherein the GIP: GLP-1 Peptide has a receptor agonist potency ratio that is 2.5:1 to about 5:1 GIP to GLP-1.

25. The method of any one of claims 1-23, wherein the GIP: GLP-1 Peptide has a receptor agonist potency ratio that is 2.5:1 to about 3.5:1 GIP to GLP-1.

26. A GIP: GLP-1 Peptide, or a pharmaceutically acceptable salt thereof, for use in treating type 2 diabetes in a patient in need thereof wherein the use comprises:
a) administration of a titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a maintenance dose of that GIP: GLP-1 Peptide;
wherein the titration dose is about 50% of the maintenance dose.

27. A GIP: GLP-1 Peptide, or a pharmaceutically acceptable salt thereof, for use in treating type 2 diabetes in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a maintenance dose of that GIP: GLP-1 Peptide;
wherein the first titration dose is about 25% of the maintenance dose, the second titration dose is about 50% of the maintenance dose and the third titration dose is about 75% of the maintenance dose.

28. A GIP: GLP-1 Peptide, or a pharmaceutically acceptable salt thereof, for use in treating type 2 diabetes in a patient in need thereof wherein the use comprises:
a) administration of a first titration dose of a GIP: GLP-1 Peptide having a GIP: GLP-1 receptor agonist potency ratio as measured after a 60 minute incubation using a casein cAMP assay normalized against GIP and GLP-1 that is about 2.5:1 to about 10:1 for a minimum of about two weeks; and thereafter
b) administration of a second titration dose of that GIP: GLP-1 Peptide for a minimum of about two weeks; and thereafter
c) administration of a third titration dose of that GIP: GLP-1 Peptide for a minimum of about two weeks; and thereafter
d) administration of a fourth titration dose of that GIP: GLP-1 Peptide for a minimum of about two weeks; and thereafter
e) administration of a fifth titration dose of that GIP: GLP-1 Peptide for a minimum of about two weeks; and thereafter
f) administration of a maintenance dose of that GIP: GLP-1 Peptide;
wherein the first titration dose is about 17% of the maintenance dose, the second titration dose is about 33% of the maintenance dose, the third titration dose is about 50% of the maintenance dose, the fourth titration is about 66% of the maintenance dose and the fifth titration dose is about 83% of the maintenance dose.

28. A composition comprising a GIP: GLP-1 Peptide having GIP: GLP-1 receptor agonist potency ratio as measured after a 60 minute incubation using casein cAMP assay normalized against GIP and GLP-1 that is about 2.5:1 to about 10:1; and a pharmaceutically acceptable excipient.

29. A composition as claimed by claim 28 wherein the composition is administered at least two weeks as an escalation dose.

30. A composition as claimed by claim 28 or 29 wherein the composition is administered at least four weeks as an escalation dose.

31. A composition as claimed by any one of claims 28 to 30 wherein the composition is administered as a maintenance dose for at least two weeks.
